(19) United States
(12) Patent Application Publication
(10) Pub. No.: US 2026/0198850 A1
(43) Pub. Date: Jul. 16, 2026

(54) TECHNIQUES FOR EVALUATING AMBIENT LIGHT EXPOSURE AND CHRONOTYPE VIA WEARABLE DEVICES

(71) Applicant: Oura Health Oy, Oulu (FI)

(72) Inventors: Olli-Pekka Puolitaival, Oulu (FI); Mari Pauliina Karsikas, Oulu (FI); Mikko Pellervo Tuohimaa, Kempele (FI); Mats Kyyrö, Oulu (FI); Anu Minna Kaarina Pramila, Oulu (FI); Matias Valtteri Kukka, Tampere (FI)

(21) Appl. No.: 19/446,596

(22) Filed: Jan. 12, 2026

Related U.S. Application Data
(60) Provisional application No. 63/744,589, filed on Jan. 13, 2025.

(51) Int. Cl. A61B 5/00; G01J 1/42; H05B 47/11

(52) U.S. Cl. CPC A61B 5/486; A61B 5/6826; A61B 5/742; A61B 5/746; G01J 1/4204; H05B 47/11; A61B 2560/0242

(57) ABSTRACT

Methods, systems, and devices for evaluating ambient light exposure and chronotype are described. The system may perform one or more physiological measurements associated with physiological data of a user and perform one or more measurements of an environmental light exposure (e.g., ambient light exposure) of the user. The system may determine one or more physiological effects of an environment of the user on the physiological data. In some cases, the system may transmit one or more signals to cause an external device to perform one or more actions to adjust the environmental light exposure of the user within the environment based on determining the one or more physiological effects.

### CROSS REFERENCE

The present Application for Patent claims priority to U.S. Provisional Patent Application No. 63/744,589 by Puolitaival et al., entitled “TECHNIQUES FOR EVALUATING AMBIENT LIGHT EXPOSURE AND CHRONOTYPE VIA WEARABLE DEVICES,” filed January 13, 2025, which is assigned to the assignee hereof and expressly incorporated by reference herein.

### FIELD OF TECHNOLOGY

The following relates to wearable devices and data processing, including techniques for evaluating ambient light exposure and chronotype via wearable devices.

### BACKGROUND

Some wearable devices may be configured to collect data from users associated with the heart rate of the user, such as motion data, temperature data, photoplethysmogram (PPG) data, etc. However, health-related insights regarding collected physiological data may be of little utility in cases where users do not view or act in accordance with the health-related insights. As such, some techniques for collecting and displaying physiological data may be improved.

### BRIEF DESCRIPTION OF THE DRAWINGS

FIG. 1 shows an example of a system that supports techniques for evaluating ambient light exposure and chronotype via wearable devices in accordance with aspects of the present disclosure.

FIG. 2 shows an example of a graphical user interface (GUI) that supports techniques for evaluating ambient light exposure and chronotype via wearable devices in accordance with aspects of the present disclosure.

FIG. 3 illustrates an example of a system that supports techniques for evaluating ambient light exposure and chronotype via wearable devices in accordance with aspects of the present disclosure.

FIG. 4 illustrates an example of a system that supports techniques for evaluating ambient light exposure and chronotype via wearable devices in accordance with aspects of the present disclosure.

FIG. 5 shows a block diagram of an apparatus that supports techniques for evaluating ambient light exposure and chronotype via wearable devices in accordance with aspects of the present disclosure.

FIG. 6 shows a block diagram of a wearable device manager that supports techniques for evaluating ambient light exposure and chronotype via wearable devices in accordance with aspects of the present disclosure.

FIG. 7 shows a diagram of a system including a device that supports techniques for evaluating ambient light exposure and chronotype via wearable devices in accordance with aspects of the present disclosure.

FIGS. 8 and 9 show flowcharts illustrating methods that support techniques for evaluating ambient light exposure and chronotype via wearable devices in accordance with aspects of the present disclosure.

### DETAILED DESCRIPTION

Some wearable devices may be configured to collect physiological data from users, including temperature data, heart rate data, heart rate variability (HRV) data, sleep data, respiratory data, and the like. Acquired physiological data may be used to analyze behavioral and physiological characteristics associated with the user, such as movement, sleep patterns, activity patterns, and the like. Many users have a desire for more insight regarding their physical health, including their sleeping patterns, activity, and overall physical well-being. In particular, many users may have a desire for more insight regarding their circadian rhythm and chronotype.

A user’s chronotype refers to the user’s natural tendency or inclination to sleep at certain times, and be awake at other times. Different users may have different chronotypes, such as an “early bird” chronotype and a “night owl” chronotype. Similarly, a user’s circadian rhythm may refer to a natural, internal process that regulates an individual’s sleep-wake cycle, that repeats approximately every 24 hours. Ideally, a user’s circadian rhythm (e.g., their 24-hour sleeping/waking schedule) is in-line with their chronotype. A user’s chronotype and circadian rhythm may be affected by biometric and genetic factors, as well as environmental factors. For example, exposure to environmental light may affect a user’s hormone levels, which may impact the user’s inclination to sleep at certain times. Environmental light may include “natural” light from the sun and “artificial” light from indoor lighting. Environmental light levels may vary throughout the day and evening, depending on personal habits and location. For example, a user in the Northern Hemisphere may experience very little natural light during winter months. Differences in environmental light may affect user activities, such as sleep. In some cases, a user may experience negative side-effects (such as difficulties sleeping) when the user’s light exposure throughout the day is out of sync with their chronotype. For example, a user that is exposed to too much environmental light shortly before bedtime may find it difficult to fall and stay asleep.

However, health-related insights regarding collected physiological data may be of little utility in cases where users do not view or act in accordance with the health-related insights. For example, a wearable device may collect physiological data that indicates a user experiencing negative side-effects (such as difficulties sleeping) when the user’s light exposure throughout the day is out of sync with their chronotype/circadian rhythm, and may display messages indicating the difficulties sleeping to the user. In this example, the collected physiological data and related messages (e.g., sleep difficulties messages) may be of little utility if the user does not view the message, or if the user does not take any actions to adjust their behaviors or environment in an attempt to improve their sleep difficulties. In other words, the physiological data, on its own, may be of little value if the collected physiological data is not accompanied with some action that is taken in response to the collected physiological data.

Aspects of the present disclosure are directed to techniques for evaluating a user’s exposure to environmental light using photodetectors of a wearable device and providing alerts or recommendations to the user when the user’s environmental light exposure is out of sync with their chronotype/circadian rhythm, or otherwise affecting their sleeping patterns or other physiological data. In particular, photodetectors of a wearable device may be used to detect environmental light (e.g., ambient light) when the photodetectors are not being used to perform photoplethysmography (PPG) measurements. In some cases, photodetectors of a wearable device may be used to detect environmental light (e.g., ambient light) when the photodetectors are also being used to perform PPG measurements. In other words, in some cases, photodetectors may be used to simultaneously perform PPG measurements and measure environmental light. As such, a wearable device may collect ambient light measurements and determine a quantity of environmental light experienced by the user based on the ambient light measurements. For the purposes of the present disclosure, the term “ambient light” may include any light received by the light-detecting components (e.g., photodetectors) of the wearable device that is not attributable to light transmitted by a light-emitting component of the wearable device. Thus, ambient light may refer to artificial indoor lighting (e.g., lamps, office lighting, sunlight, and the like).

In some cases, the wearable device may evaluate the user’s environmental light exposure relative to their physiological data (e.g., relative to their circadian rhythm/chronotype) and provide alerts or recommendations to the user that include information regarding how the user’s environmental light exposure may have affected their physiological data (and/or how the user’s environmental light exposure is currently affecting or is likely to affect the user’s physiological data in the future). For example, a wearable system may indicate that the user is experiencing too much light shortly before bedtime and provide a recommendation to limit light exposure leading up to bedtime in order to improve sleep quality. In some examples, the wearable system may indicate that the user is experiencing too much light during sleep and provide a recommendation to limit light exposure during sleep in order to improve sleep quality. In other examples, the wearable system may indicate that the user is not experiencing enough light when the user is awake, and may therefore provide a recommendation to increase light exposure when the user is awake.

In some cases, the environmental light exposure may be used to adjust the user’s environment, such as by triggering lights to adjust exposure, dimming lights as bedtime approaches, drawing blinds or curtains, or otherwise adjusting a sleep schedule of the user. Additionally, the environmental light exposure may be used to determine a skincare index by estimating a level of ultraviolet (UV) exposure, as well as using other data such as location (e.g., to determine a UV index), hydration, sleep, and stress data. In this regard, aspects of the present disclosure may enable a system to selectively adjust characteristics of a user’s surrounding environment (e.g., light) based on acquired physiological data in order to improve a user’s sleep and overall health.

Aspects of the disclosure are initially described in the context of systems supporting physiological data collection from users via wearable devices. Aspects are then described with reference to an example graphical user interface (GUI). Aspects of the disclosure are further illustrated by and described with reference to apparatus diagrams, system diagrams, and flowcharts that relate to techniques for evaluating ambient light exposure and chronotype via wearable devices.

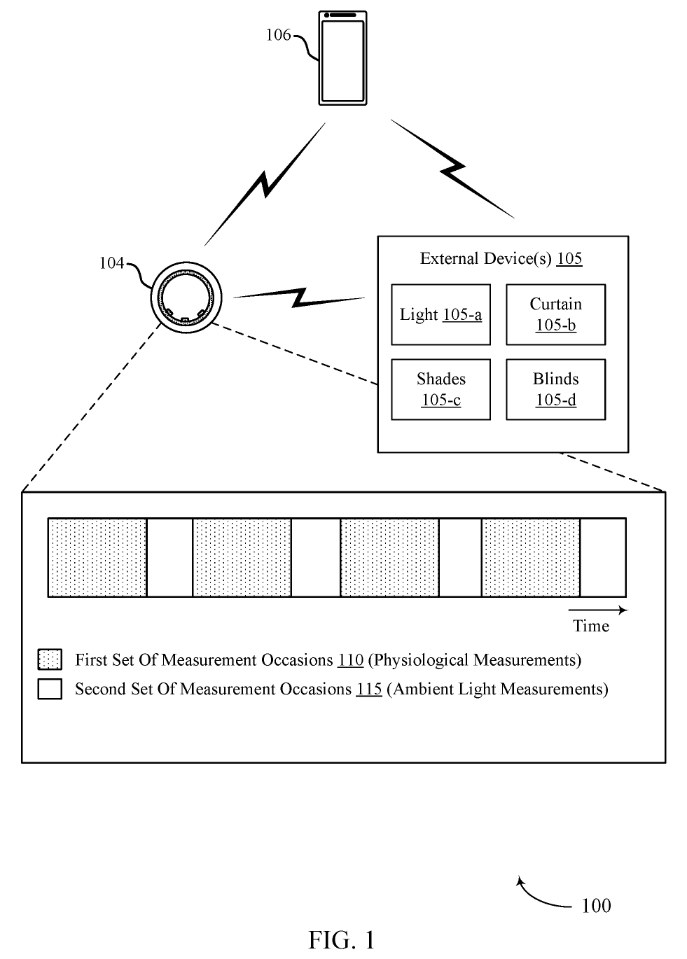

FIG. 1 shows an example of a system 100 that supports techniques for evaluating ambient light exposure and chronotype via wearable devices in accordance with aspects of the present disclosure. The system 100 illustrates an example of a wearable device 104 (e.g., wearable ring device 104), one or more external devices 105, and a user device 106, as described with reference to FIG. 1.

As described with reference to FIG. 1, the wearable device 104 may be worn by a user and may determine one or more effects of an environment of the user on the physiological data (e.g., user skin temperature, pulse waveforms, respiratory rate, heart rate, HRV, blood oxygen levels, and the like). For example, the wearable device 104 may perform, using one or more physiological sensors (e.g., one or more light-receiving components of the wearable device 104), one or more physiological measurements associated with physiological data of a user. In some examples, the one or more physiological sensors may generate physiological signal data of the user in which the physiological signal data may be indicative of (or utilized to generate) physiological measurements or data. In other words, the one or more physiological sensors may output (e.g., via one or more signals) one or more indications of measurements that may be utilized to generate or determine physiological data associated with the user. In some examples, the one or more light-receiving components may be an example of photodetectors, photosensors, and the like, and the one or more physiological measurements may be an example of PPG measurements.

For example, the wearable device 104 may perform physiological measurements during a first set of measurement occasions 110. The first set of measurement occasions 110 may be an example of a duration of time that the one or more physiological measurements associated with physiological data of a user are performed. The one or more physiological measurements are performed using the one or more light-receiving components and one or more light-transmitting components of the wearable device 104 during the first set of measurement occasions 110 at the wearable device. The one or more light-transmitting components may be an example of LEDs, vertical-cavity surface-emitting lasers (VSCELs), laser diodes, and the like. The one or more light-receiving components may be positioned on an inner curved surface of the wearable device 104. In some cases, the inner curved surface may be configured to at least partially contact a tissue of the user when the wearable device 104 is worn by the user.

The wearable device 104 may collect one or more measurements of an environmental light exposure associated with the user based on measuring an amount of ambient light received at the photodetectors (e.g., one or more light-sensing components) of the wearable device 104. For example, the wearable device 104 may perform, using the one or more light-receiving components of the wearable device 104, one or more measurements of an environmental light exposure (e.g., ambient light incident on the wearable device that is not attributable to light emitted by the wearable device) of the user. In some examples, the one or more light-sensing components may generate environmental light data indicative of ambient light. The one or more measurements of the environmental light exposure may be performed while the wearable device 104 is being worn by the user. In such cases, the wearable device 104 may perform the measurements of environmental light exposure during a second set of measurement occasions 115. The second set of measurement occasions 115 may be an example of a duration of time that the measurements of environmental light exposure are performed. In such cases, the one or more measurements of the environmental light exposure are performed during the second set of measurement occasions 115.

The second set of measurement occasions 115 may be non-overlapping in time with respect to the first set of measurement occasions 110. For example, the PPG measurements (e.g., physiological measurements performed during the first set of measurement occasions 110) and the ambient light measurements (e.g., measurements of environmental light exposure performed during the second set of measurement occasions 115) may be performed at different times (e.g., during different measurement occasions). In some cases, the first set of measurement occasions 110 may alternate in time with the second set of measurement occasions 115 without overlapping (e.g., non-overlapping in the time domain). In such cases, the wearable device 104 may collect environmental light data via photodetectors during the second set of measurement occasions 115 while the wearable device’s 104 LEDs are powered off, thereby measuring ambient light during the second set of measurement occasions 115 when the wearable device 104 is not measuring PPG. In additional or alternative implementations, the first and second sets of measurement occasions may at least partially overlap with one another in the time domain.

In some cases, the one or more measurements of the environmental light exposure and the physiological measurements may be performed during a same measurement occasion. That is, in some cases, the photodetectors of the wearable device may be used to simultaneously perform PPG measurements and environmental light measurements. For example, the wearable device 104 may perform the physiological measurements and the one or more measurements of the environmental light exposure during the first set of measurement occasions 110. In such cases, the second set of measurement occasions 115 may be omitted, and/or the second set of measurement occasions 115 may be used to perform other measurements by the wearable device 104. For example, the PPG measurements and the ambient light measurements may be performed at the same times (e.g., during the same measurement occasions).

In some examples, the wearable device 104 may further include a communication interface. The communication interface may allow for one or more components of the wearable device 104 to communicate with one or more other components of the wearable device 104. The communication interface may also allow the wearable device 104 to communicate with the user device 106, the one or more external devices 105, or other devices. In some examples, the wearable device 104 may transmit one or more signals to the user device 106 indicative of information to display to the user (e.g., an alert, recommendation, or instruction). Additionally, or alternatively, the wearable device may transmit a signal (e.g., a control signal) to at least one external device of the one or more external devices 105 to cause an adjustment to the ambient light in the environment of a user.

In some cases, the system 100 may further include one or more processors operatively coupled to the wearable device 104, the user device 106, or both. The user device 106 may be communicatively coupled to the wearable device 104. The system 100 may utilize the one or more processors to perform one or more operations (e.g., obtaining data, determining metrics, etc.).

In some aspects, the respective devices of the system 100 may support techniques for operating the LEDs at lower power levels (or completely turning the LEDs off) by using ambient light as an additional and/or alternative light source as compared to using only LEDs (or other light sources within the wearable device 104). That is, the wearable device 104 may use ambient light external from the wearable device 104 (e.g., sunlight, indoor fluorescent lights, etc.) as a signal source to capture PPG signals or as an additional source to allow lowering the power outputted by the LEDs. In some cases, using ambient light external from the wearable device 104 as the signal source to capture PPG signals may maximize the photodetector signals.

The wearable device may determine the strength and/or quality of the signal (e.g., the PPG signal) that is derived from the ambient light and determine whether to deactivate the LEDs and use the ambient light as the single light source or, alternatively, to operate the LEDs at a lower power level and refrain from using the ambient light as a light source. By dynamically adjusting the power output of the LEDs based on real-time signal quality metrics, techniques described herein may perform the same physiological measurements while consuming less power, which may lead to more accurate measurements. In such cases, utilizing ambient light as an external light source and optimizing the LED power based on one or more quality metrics may decrease a power consumption at the wearable device, which may lead to longer battery life.

For example, wearable device 104 of the system 100 may measure, at the photodetector, a PPG signal derived from an ambient light source that is received external to the wearable device 104 (e.g., an alternative light source other than the LED). The system 100 may calculate a quality metric of the PPG signal after measuring the PPG signal. In such cases, the system 100 may adjust the power output level of the LED based on the quality metric of the PPG signal.

In some examples, the wearable device 104 and the user device 106 may communicate with one another via one or more wireless connections. For instance, the wearable device 104 may send the physiological data to the user device 106, and the user device 106 may send firmware/configuration updates to the wearable device 104.

In some examples, the wearable device 104, the user device 106, or both may determine one or more physiological effects (and/or predicted/future physiological effects) of an environment of the user on the physiological data based on performing the one or more measurements of the environmental light exposure during the second set of measurement occasions 115. That is, the system 100 may determine how the user’s exposure to ambient/environmental light affects their physiological data and overall health. Physiological effects which may be identified, classified, and/or predicted via the system 100 may include any physiological effects known in the art, including physiological effects associated with falling asleep, physiological effects associated with waking up, physiological effects associated with anxiety, stress, relaxation, and the like. In some cases, the physiological effects may be associated with a skin health of the user.

In some aspects, the system 100 may determine physiological effects or predicted physiological effects utilizing a model (e.g., machine learning model, classifier, or algorithm). The system 100 may utilize one or more metrics (e.g., an ambient light exposure metric and a physiological timing metric) as input to a machine learning model to determine or predict physiological effects on the user caused by or associated with the environmental light exposure. For example, based on measuring relatively high ambient light exposure during the night and a reduced amount of sleep, the system 100 may predict tiredness and lethargy for the following day.

In some aspects, the system 100 may determine and/or predict physiological effects that ambient/environmental light has on the user’s overall health and physiological data by comparing collected data to baseline data associated with the user. For example, the system 100 may collect baseline physiological data from the user over time, as well as baseline ambient light data corresponding to the baseline physiological data. Subsequently, the system 100 may identify a change in the amount of ambient/environmental light that the user receives over time (e.g., change relative to the baseline ambient light data), and may compare additional physiological data collected at the same time (or subsequent) to the change in ambient light to the user’s baseline physiological data in order to determine what kind of effect the change in the ambient light data had on the user’s physiological data and/or overall health.

In some examples, the baseline physiological data associated with the user may include both nighttime baseline physiological data and daytime baseline physiological data. For example, the nighttime baseline physiological data may be determined based on the median ambient light received during a sleeping period for a user (e.g., the median amount of light in the environment of the user while they are asleep). In other examples, the daytime physiological data may be determined based on the median ambient light received during an awake period for a user.

In further examples, the system 100 may adjust the baseline physiological data associated with the user based on identifying a change in the amount of ambient/environmental light that the user receives throughout a duration that satisfies a threshold. For example, the system 100 may identify a decrease in the amount of ambient/environmental light that the user receives that satisfies a threshold based on the duration the decrease is sustained (e.g., the user receives less light for a period of time greater than two weeks). The change in the amount of ambient/environmental light may indicate a change in user habits (e.g., bedtime/wake-up routine) or the user’s environment (e.g., a change in season). In response, the system 100 may adjust the baseline physiological data to better identify changes in the amount of ambient/environmental light that the user receives as the unadjusted baseline may lead to the system 100 comparing additional physiological data to inaccurate baseline physiological data for the user.

In some examples, based at least in part on determining the physiological effects of the environment (e.g., ambient/environmental light), an intervention may occur. For example, based at least in part on determining a negative effect of an ambient light condition on the overall health of the user, the wearable device 104, the user device 106, or both, generate an alert or instruction to modify the environmental light exposure of the user. In other cases, the system may cause an intervention by transmitting a control signal to at least one external device (e.g., external devices 105) to adjust the environment of the user. In particular, the system may cause an intervention by causing an external device to adjust one or more operational parameters of the device in such a manner as to modify the environmental light exposure of the user.

In some cases, determined effects of ambient light on the user’s physiological data and/or overall health may be used to modify or adjust one or more algorithms used to determine various physiological states, metrics, and/or scores (e.g., Sleep Score, Activity Score, Readiness Score) associated with the user. Such modifications based on effects of ambient light may be used to further improve the accuracy and/or reliability of such algorithms. For example, the system 100 may determine that a decrease in ambient light exposure for a particular day results in a change in the user’s HRV metrics for that day and/or a subsequent day. In such cases, the user may still exhibit high “readiness” despite the changed HRV metric due to the change in the ambient light exposure. As such, the system 100 may use this determined effect to adjust the algorithm that is used to calculate the user’s Readiness Score for that day (and/or subsequent days). In this regard, the system 100 may account for the change in the ambient light exposure when calculating the user’s Readiness Score, thereby resulting in a Readiness Score that more accurately reflects the user’s readiness and ability to perform activities and other tasks.

In some examples, the system 100 may determine a user physiological timing metric based at least in part on physiological measurements determined from the physiological signal data. The term “user physiological timing metric” and like terms may be used to refer to parameters or metrics that relate to the user’s circadian rhythm and/or chronotype. The user physiological timing metric may comprise at least one of a circadian rhythm metric (e.g., midpoint of a user’s circadian rhythm, or relative timing of a user’s circadian rhythm relative to a 24-hour day), a chronotype classification (e.g., “early bird,” “night owl,” etc.), a sleep window metric (e.g., optimal or recommended sleep window, bedtime, wake time, etc.), a circadian phase estimate (e.g., estimate of where a user is within their circadian rhythm), or the like.

For example, the system 100 may determine the physiological timing metric (e.g., a chronotype classification, circadian rhythm, sleep window metric, and/or circadian phase estimate) of the user based on the one or more physiological measurements performed during the first set of measurement occasions. The system 100 may determine a timing of the environmental light exposure relative to the chronotype based on the one or more measurements of the environmental light exposure. For example, the system 100 may determine that the user receives increased amounts of sunlight during the morning hours based on the user being identified as an “early bird” chronotype. The one or more physiological effects of the environment may be based on the timing of the environmental light exposure relative to the chronotype (e.g., based on whether the timing of the user’s ambient light exposure is in sync with the user’s chronotype/circadian rhythm or not). In some examples, the system 100 may utilize the timing of the environmental light exposure relative to the chronotype to determine the user physiological timing metric. For example, the system 100 may determine that exposure to environmental light may affect a user’s hormone levels, which may impact the user’s inclination to sleep at certain times based on the determined chronotype and the amount of ambient light the user is exposed to. In such cases, the system 100 may compare the user’s environmental light exposure to the user’s chronotype and make recommendations to the user accordingly.

For example, the wearable device 104 may transmit one or more signals to the user device 106 to cause the user device 106 to display instructions for modifying the timing of the environmental light exposure based on the chronotype of the user. In such cases, the system 100 may provide recommendations to the user on how to adjust environmental light exposure based on the user’s determined chronotype (e.g., “Try to avoid light exposure in the hour leading up to bedtime to improve your sleep,” or “Aim for 20 minutes of sunlight first thing in the morning to get a jump-start on your day”). The one or more signals may be configured to cause the user device 106 to display instructions for modifying the timing of the environmental light exposure based on the chronotype of the user. For example, the system 100 may provide recommendations to the user to increase an amount of sunlight during the day due to the user being classified as a “night owl” chronotype. In such cases, the system 100 may transmit instructions for one or more external devices 105 to open the blinds 105-d, shades 105-c, and/or open the curtain 105-b to let sunlight into the indoor spaces.

For the purposes of the present disclosure, the term “circadian rhythm chronotype,” “chronotype,” “or “circadian rhythm,” and like terms, may be used to refer to an individual circadian rhythmicity, which is related to sleep, diet, physical activity patterns, and the like. The circadian rhythm is a biological, internal process running in the background of daily functions and orchestrating a twenty-four-hour cycle (or an approximately twenty-four-hour cycle) in the users. The circadian rhythm regulates biological functions and processes, including but not limited to sleep-wake cycle, alertness, digestion, body temperature, hormone release, and the like. In some cases, a user’s circadian rhythm (e.g., body clock) may be externally sensitive and influenced by lifestyle choices and other factors. For example, exposure to the light at different times of the day may be an example that influences the internal body clock.

In some cases, the system 100 may determine a sleep regularity index. The sleep regularity index may measure the consistency of a user’s sleep timeline and their pattern of light/dark exposure. In some cases, the user may experience misalignment between the user’s circadian rhythm and/or chronotype and the sleep/wake cycle. Irregular sleep may decrease a user’s daily performance, and cognitive functions, and is associated with health threatening risk factors. In such cases, having a regular sleep pattern and a regular pattern of light/dark exposure may be beneficial to the overall health of the user. In some cases, compliance with a user’s established sleep pattern and light/dark exposure may be a contributing factor to the user’s sleep quality. As described herein with reference to FIG. 1, the system 100 may transmit one or more signals to cause an external device 105 to perform one or more actions external to the wearable device 104 to adjust one or more characteristics associated with the environment of the user, such as a light therapy assist, for example. In some cases, the system 100 may transmit one or more signals to cause the user device 106 to display information associated with one or more physiological effects of an environment of the user on the physiological data.

In some cases, the system 100 may receive geographical location data associated with a location of the user during a time interval of the one or more measurements of an environmental light exposure (e.g., during the second set of measurement occasions 115). In such cases, the system 100 may determine a UV light exposure of the user based on the geographical location data and the one or more measurements of an environmental light exposure. For example, a user in the Northern Hemisphere may experience an abundance of natural light during summer months. In such cases, based on the geographical location data, the system 100 may instruct one or more external devices 105 to close the shades 105-c, blinds 105-d, and/or close the curtain 105-b during nighttime hours to block out the natural light when the user’s light exposure throughout the evening and/or night is out of sync with their chronotype. Differences in environmental light may affect user activities, such as sleep. In some cases, a user may experience negative side-effects (such as difficulties sleeping) when the user’s light exposure throughout the day is out of sync with their chronotype.

In some examples, the system 100 may further use the geographical location data associated with the location of the user during a time interval of the one or more measurements of an environmental light exposure (e.g., during the second set of measurement occasions 115) to determine one or more weather patterns. The system 100 may determine a UV light exposure of the user based on the one or more weather patterns associated with the geographical location data and the one or more measurements of an environmental light exposure. For example, a user in a relatively warm location may remain indoors during the summer months. In such cases, based on the one or more weather patterns associated with the geographical location data, the system 100 may instruct one or more external devices 105 to open the shades 105-c, blinds 105-d, and/or open the curtain 105-b during daytime hours to increase the natural ambient light when the user’s light exposure throughout the day is out of sync with their chronotype (e.g., the user is not receiving enough light during the daytime due to the warm weather).

The system 100 may determine a skin health metric that is based on the environmental light exposure (e.g., UV light exposure). In such cases, the system 100 may use location data to estimate UV exposure, which is used to calculate the skin health metric. Using the user’s location may improve estimations of ambient light exposure and thus provide improved and tailored recommendations to the user to adjust ambient light exposure. For example, the system 100 may determine that the user is at the beach using the location data and estimate the UV light exposure based on the location and time data (e.g., at the beach at 11:00 AM). In such cases, the system may provide recommendations to the user to go inside during the high UV index times of the day and/or apply sunscreen at regular intervals if the user is outside during those times of the day.

In some cases, the photodetectors of the wearable device 104 may be able to differentiate between different colors and/or wavelengths of light. For example, the system 100 may determine at least a first wavelength exposure and a second wavelength exposure of the user based on the one or more measurements of the environmental light exposure performed during the second set of measurement occasions 115. The one or more physiological effects may be based on the first wavelength exposure, the second wavelength exposure, or both. In this regard, and depending on the types and/or sensitivity of the photodetectors or other light-receiving components of the wearable device 104, the wearable device 104 may be able to identify (and measure) the user’s exposure to different types/wavelengths of light, such as different wavelengths of visible light, ultraviolet (UV) light (e.g., UV-B light), etc. Moreover, in some aspects, the wearable device 104 may be able to measure various characteristics of the user’s exposure to the respective types/wavelengths of light, such as a total amount of exposure (e.g., light “dosage” metric), an exposure duration, an intensity of exposure, etc.

In some aspects, the system 100 may be configured to evaluate one or more exposure criterion of the user for the one or more types/wavelengths of light. The exposure criterion may include various thresholds that are configured to prevent the user from experiencing detrimental effects or harm based on the user’s environmental light exposure, and/or thresholds that are configured to ensure that the user is receiving sufficient health-related benefits as a result of their environmental light exposure. Exposure criterion may include a threshold amount of light exposure to a specific wavelength(s) (e.g., total “dosage” of light exposure), a threshold intensity of light exposure to a specific wavelength(s), a threshold time duration of light exposure to specific wavelength(s), etc. In some aspects, the system 100 may perform certain actions based on a satisfaction (or lack thereof) of the one or more exposure criterion, such as issuing alerts or messages to the user, adjusting external devices, and the like.

In such cases, the system 100 may be able to determine whether the user was indoors or outdoors based on the color/wavelength of ambient light. For example, the system 100 may be able to determine that the user was indoors based on determining that the ambient light is fluorescent light. In other examples, the system 100 may be able to determine that the user was outdoors based on determining that the ambient light is UV light. In such cases, the system 100 may determine a location of the user (e.g., whether the user is indoors or outdoors) for at least a portion of a time interval associated with the one or more measurements of the environmental light exposure based on the first wavelength exposure, the second wavelength exposure, or both. That is, the system 100 may determine a location of the user for at least at a portion of the second set of measurement occasions 115.

In some aspects, the system 100 may be configured to selectively control/adjust one or more external devices 105 (e.g., at least one external device) based on physiological data collected via the wearable device 104, physiological effects identified based on the collected physiological data, or any combination thereof. For example, as shown in FIG. 1, the system 100 may further include one or more external devices 105 in communication with the user device 106, the wearable device 104, or both. The external devices 105 may include, but are not limited to, a light 105-a, a curtain 105-b, shades 105-c, and/or blinds 105-d. Additionally, or alternatively, the external devices 105 may include any external device capable of communicating with the user device 106. For example, additional external devices 105 may include any smart device, such as a virtual assistant technology device, a smart home device, and the like.

In some aspects, the one or more external devices 105 may be configured to modify one or more operational parameters associated with the respective external device 105, for example, based on signals or instructions received from the user device 106, the wearable device 104, or both. Further, the one or more external devices 105 may change the environment in which the respective external device 105 is placed (e.g., modify a surrounding environment associated with the user corresponding to the wearable device 104) based on modifying the one or more operational parameters. For example, the light 105-a, curtain 105-b, shades 105-c, and/or blinds 105-d may adjust the brightness of the surrounding environment (e.g., increase the brightness or decrease the brightness). That is, the system 100 may be configured to cause the external devices 105 to perform actions that may adjust the user’s ambient light exposure.

In some examples, the user device 106 may transmit instructions to the one or more external devices 105 to adjust one or more characteristics associated with the environment of the user based at least in part on determining the one or more physiological effects. Additionally, the one or more external devices 105 may modify the operational parameters based on receiving the instructions that are sent by the user device 106. For instance, the light 105-a may power off (e.g., enter a low power state) based on receiving instructions to do so from the user device 106. In some cases, the system 100 may transmit one or more signals to cause the user device 106 to display information associated with one or more physiological effects of the environment of the user.

In some examples, the user device 106 may instruct the one or more external devices 105 to adjust operational parameters based on receiving physiological data from the wearable device 104. Additionally, or alternatively, the user device 106 may instruct the one or more external devices 105 to adjust the environmental light exposure of the user within the environment based on determining the one or more physiological effects of the environment of the user on the physiological data. In other words, the physiological data collected by the wearable device 104 and acquired by the user device 106 may be used by the user device 106 to control the one or more external devices 105.

For instance, the user device 106 may receive the physiological data collected by the wearable device 104, identify that the user is falling asleep based on the received physiological data, and transmit an instruction to the light 105-a, curtain 105-b, shades 105-c, and/or blinds 105-d. The instructions transmitted by the user device 106 may be configured to adjust operational parameters of the light 105-a, curtain 105-b, shades 105-c, and/or blinds 105-d. For instance, the transmitted instructions may include a configuration that may cause the light 105-a to dim the brightness of the light 105-a, draw the curtain 105-b, and close the shades 105-c and/or blinds 105-d.

By way of another example, if the system 100 identifies a user is experiencing negative side-effects (such as difficulties waking up in the morning) when the user’s light exposure throughout the day is out of sync with their chronotype, the system 100 may adjust the one or more external devices 105 to open the curtain 105-b, shades 105-c, and/or blinds 105-d to facilitate letting natural light in to expose the user to natural light and align the circadian rhythm. Moreover, the system 100 may be configured to adjust operational parameters of the external devices such as the light 105-a based on physiological effects (e.g., difficulties waking up in the morning), such as adjusting the light 105-a to increase brightness when the psychological data suggests that the user is still sleeping.

In some examples, the user may be able to manually input certain actions that they want to occur in accordance with the physiological states and/or physiological effects identified by the user device 106. In other words, a user may be able to input rules or commands which define certain “relationships” between operational parameters and identified physiological effects (e.g., “If ring detects X physiological effect, adjust Y operational parameter of external device Z.”). By receiving input from the user, the user device 106 may execute the customized modifications to be made to the operation of the one or more external devices 105 upon identifying a specified physiological effect of the user.

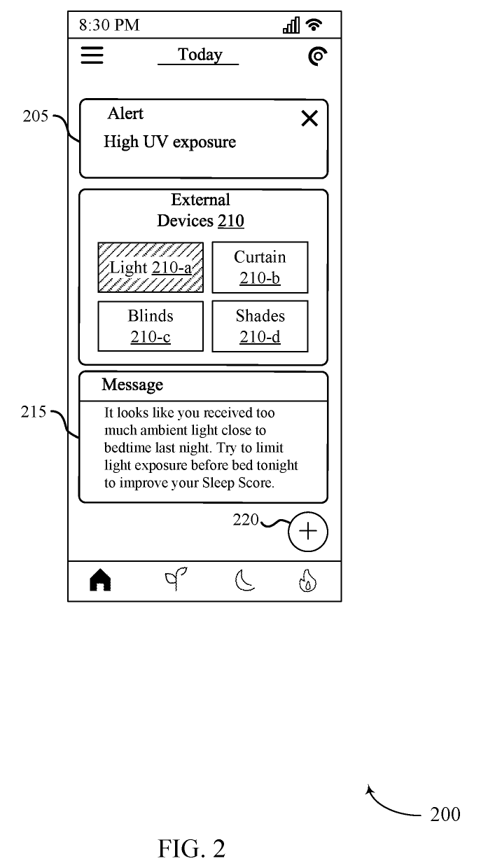

For example, the user may manually input into the GUI of the user device 106 that they want the blinds 105-d to be opened at sunrise if the wearable device 104 detects that the user is experiencing a circadian rhythm misalignment from sleeping in too late (e.g., if the wearable device 104 detects a physiological effect associated with physiological data). Accordingly, the user device 106 may transmit an instruction to the blinds 105-d to open at sunrise if the user device 106 identifies that the user is still asleep based on the physiological data collected by the user device 106. By way of another example, the user may manually input into the GUI of the user device 106 that they want the light 105-a to be turned off if the wearable device 104 detects that the user is falling asleep. Thus, the user device 106 may tailor the one or more external devices 105 to operate based on the user’s desires to occur in correlation with the user’s identified physiological effects. This may be further understood with reference to FIG. 2.

In some aspects, the system 100 may optimize the environment of the room in which the external devices 105 are placed to achieve surrounding conditions which lead to a healthier lifestyle, a more efficient use of resources, and a safer environment. In some examples, the system 100 may improve the health of the user wearing the wearable device 104 by inducing the conditions which lead to better sleep (e.g., lower light). Furthermore, the system 100 may reduce electrical consumption by turning off one or more of the one or more external devices 105 when the one or more external devices 105 are no longer being used by the user (e.g., turning off a light 105-a when the user is no longer awake).

FIG. 2 shows an example of a GUI 200 that supports techniques for evaluating ambient light exposure and chronotype via wearable devices in accordance with aspects of the present disclosure. The GUI 200 may implement, or be implemented by, aspects of the system 100. For example, the GUI 200 may include an example of a GUI that may be displayed via the user device 106 illustrated in FIG. 1.

In some aspects, a server of the system (e.g., server communicatively coupled with the wearable device 104 and/or user device 106) may cause the GUI 200 of the user device 106 (e.g., mobile device) to display options for user input 220 regarding operational preferences for one or more external devices 210 in accordance with identified physiological effects. In such cases, the system may output the user’s customizable preferences for the one or more external devices 210 on the GUI 200 of the user device to indicate the ways in which the user may selectively modify the operational parameters of the one or more external devices 210 in accordance with an identified physiological effect(s). In other words, the system may receive (e.g., via the GUI 200) user inputs 220 associated with relationships between physiological effects and operational parameters of external devices 210. For example, the user may input relationships/commands, via user input 220, for adjusting the user’s environment (e.g., “If I receive too much light during the day, dim the lights.”).

Continuing with the example above, the user may be presented with the application page upon opening the wearable application. As shown in FIG. 2, the application may display external devices 210. The external devices 210 listed on the application page may include one or more external devices 210 which may receive instruction from the user device based on physiological effects identified by the user device, the wearable device, or both. The external devices 210 may include a light 210-a, a curtain 210-b, blinds 210-c, and shades 210-d. In some cases, the user may be able to add external devices 210 to the application page via user input 220 (e.g., a second light, a second curtain, etc.).

On the application page, the user may select one or more of the external devices 210 for which they would like to manually input operational parameters. For example, if the user would only like to modify an operational parameter of the light 210-a in scenarios in which the system determines that the user received too much light during the day, the user may select the light 210-a from the application page. Alternatively, if the user would like to modify operational parameters of the light 210-a, the curtain 210-b, and the blinds 210-c in scenarios in which the system determines that the user received too much light during the day, the user may select the light 210-a, the curtain 210-b, and the blinds 210-c on the application page. In cases in which the user is inputting preferences for more than one of the external devices 210, the user may select one of the external devices 210 on the application page individually, proceed to another application page to input preferences for the external device 210 that was selected, and repeat the process for all the external devices 210 that the user may wish to input operational preferences for.

The application page may display one or more relationships between identified physiological effects and operational parameters for one of the external devices 210 that was most recently selected. The user may provide user input 220 on the application indicating the one or more relationships that may be customized. For instance, if the user desires to turn on the light 210-a when the system determines that the user has not received enough light for the day, the user may open the wearable application page and select the light 210-a from the application page.

In some examples, the system may calculate an ambient light exposure metric of the user based on measurements of the environmental light exposure. The ambient light exposure metric may indicate or characterize an amount, timing, duration, spectral composition (e.g., wavelength(s)), or pattern of environmental light exposure for the user (e.g., more environmental light exposure in the morning, and less environmental light exposure in the evening, etc.). For example, the system may determine how much ambient light the user has received in a day and the user device may display, via message 215, an indication of the amount, pattern, etc. The user device may receive signals to cause the user device to display information associated with the ambient light exposure metric via message 215 and/or alert 210. For example, the message 215 may state: “It looks like you received too much ambient light close to bedtime last night. Try to limit light exposure before bed tonight to improve your Sleep Score.” Another message 215 may indicate “It seems that you haven’t stepped outside today. Take a minute to go outside and feel the sun on your face.”

In some examples, the system may use one or more context factors to determine a message 215 and/or alert 210. For example, the system may determine a message 215 based on the day of the week (e.g., a weekday or weekend). In some examples, the user device may display a message 215 to recommend the user receive an amount of ambient light aligned with the chronotype of the user based on it being a day that the user is more likely to have leisure time (e.g., a weekend) to spend outside. For example, the message 215 may state: “Take a break outside. Use your leisure time to feel the sun on your face.” In other examples, the system may determine that the user is less likely to have leisure time (e.g., a weekday) and the user device may display a message 215 at a time when the user is more likely to comply with it (e.g., during a midday break for lunch or after traditional working hours) to recommend the user receive an amount of ambient light aligned with the chronotype of the user. For example, the message 215 may state: “Done with work? Try to increase your light exposure now to improve your Sleep Score later.”

The system may calculate a skin health metric of the user based on the measurements of the environmental light exposure. For example, the system may calculate an ambient light exposure metric that represents how much light the user has received and/or the effect of the light received on the skin of the user. The user device may receive signals to cause the user device to display information associated with the skin health metric. For example, the skin health metric may be displayed via message 215 and may indicate that the body temperature of the user has increased due to an increased value of the skin health metric (indicating that the user may be experiencing a sunburn). In other examples, the message 215 may indicate “The amount of UV light you’ve received today is off the charts. Try spending a little more time inside tomorrow to give your skin a break from the sun.”

In some cases, the system may recommend vitamin D based on the ambient light exposure. In such cases, the system may calculate a dosage of vitamin D for the user based on the one or more measurements of an environmental light exposure. The system may instruct, via the user device associated with the wearable device, the user to intake the dosage of vitamin D based on identifying that a level of ambient light exposure is below a baseline level of ambient light exposure, and/or based on a determination that one or more exposure criterion are satisfied or not. For example, a user in the Northern Hemisphere may experience very little natural light during winter months, and the system may indicate, via message 215, “It looks like you are getting less natural light this time of year. Take some vitamin D to offset the effects of receiving less natural light this winter.”

In some aspects, the system 100 may calculate or estimate a level of vitamin D synthesis that is driven or caused by the user’s environmental light exposure to specific wavelength(s) of light, and may recommend specific vitamin D dosages based on the estimated level of vitamin D synthesis. The level of vitamin D synthesis may be estimated based on the duration and/or intensity of the user’s environmental light exposure, the specific wavelength(s) of light that the user was exposed to, spectral weighting factors associated with the specific wavelength(s) that the user was exposed to, and the like. In particular, different wavelengths of light may be more or less effective to drive vitamin D synthesis, and spectral weighting factors may be used to “weight” the user’s estimated level of vitamin D synthesis based on the duration and/or intensity of the various wavelengths of light. In this regard, by estimating a level of vitamin D synthesis of the user, the system 100 may be configured to determine a vitamin D status metric for the user, where the vitamin D status metric indicates whether or not the user has received or synthesized sufficient levels of vitamin D (e.g., vitamin D status metric may indicate whether the user has more than enough vitamin D, or if the user should take vitamin D supplements in order to increase their vitamin D levels). Subsequently, recommendations regarding the dosage and/or timing for ingesting vitamin D supplements that are provided to the user may be based on the estimated level of vitamin D synthesis, and/or the vitamin D status metric.

In some implementations, techniques described herein may be used to leverage data collected via external devices (e.g., user device 106, external devices 105) and/or wearable device 104 to identify location data of the user. In such cases, the GUI 200 illustrated in FIG. 2 may be configured to display messages 215 associated with the identified location. In some examples, upon determining a location that indicates the user is likely outside in direct sunlight, the system may cause the user device 106 to display a message 215 that reminds the user to go inside and/or find some shade based on the determined UV light exposure metric, the skin-age metric, or both.

In some examples, upon determining a location that indicates the user is likely in their kitchen, the system may cause the user device to display a message 215 that reminds the user to take their vitamins (e.g., vitamin D), which are likely stored in the kitchen. In this regard, aspects of the present disclosure may be used to provide more tailored, target guidance/messaging to the user based on their location, their skin health metric, their UV light exposure metric, their ambient light exposure metric, or a combination thereof, which may increase the likelihood that the user will positively act in accordance with (e.g., in response to) the guidance/messaging.

In some cases, the wearable device may evaluate the user’s environmental light exposure relative to their circadian rhythm/chronotype, and provide alerts or recommendations to the user when the user’s environmental light exposure is out of sync with their chronotype, or otherwise affecting their sleeping patterns or other physiological data. For example, the system may indicate that the user is experiencing too much light shortly before bedtime, and provide a recommendation to limit light exposure leading up to bedtime in order to improve sleep quality. In some cases, the environmental light exposure may be used to adjust the user’s environment, such as by triggering the light 210-a to adjust exposure, dimming light 210-a as bedtime approaches, drawing blinds 210-c or the curtain 210-b, or otherwise adjusting a sleep schedule of the user. In other examples, the user physiological timing metric may comprise an indication of the chronotype of the user and the relative ambient light exposure.

In some implementations, the user device and/or servers may generate alerts 205 associated with the user’s environmental light exposure relative to their circadian rhythm/chronotype which may be displayed to the user via the GUI 200. In such cases, the application page may display an indication of the user’s environmental light exposure relative to their circadian rhythm/chronotype via alert 205.

In cases where a user’s determined circadian rhythm/chronotype is misaligned with the received physiological data based on the user’s environmental light exposure, as described herein, the server may transmit an alert 205 to the user, where the alert 205 is associated with the misalignment. In particular, alerts 205 generated and displayed to the user via the GUI 200 may be associated with recommendations to return to the user’s baseline determined circadian rhythm/chronotype. In some cases, the alert 205 may display a recommendation of how to adjust their lifestyle (e.g., including the user’s environmental light exposure) on the day of the determined misalignment and/or in the days after the determined misalignment. The alerts 205 and/or messages 215 may be configurable/customizable, such that the user may receive different alerts 205 and/or messages 215 based on the environmental light exposure of the user within the environment.

In some cases, the user may take remedial action to address the misalignment prior to the system displaying the alert 205. In such cases, the system may receive physiological data associated with the remedial action, and the system may refrain from displaying the alert 205 (e.g., override the alert 205). For example, the system may transmit one or more signals to cause an external device 210 to perform one or more actions external to the wearable device 104 to adjust one or more characteristics associated with the environment of the user, thereby preventing the misalignment or reducing the misalignment. As noted previously herein, causing the external device 210 to perform one or more actions external to the wearable device 104 to adjust one or more characteristics associated with the environment of the user may be beneficial to a user’s overall health by providing metrics to the user that may enable the user to understand how behavior changes (e.g., exposure to light, etc.) may help increase the user’s overall health and reduce an occurrence of circadian rhythm chronotype misalignment.

In some cases, the system may transmit one or more signals to cause the user device 106 to display information associated with the one or more physiological effects associated with the environment of the user, thereby preventing the misalignment or reducing the misalignment between the user’s environmental exposure and the user’s physiological characteristics (e.g., chronotype, sleeping habits, etc.). The one or more signals may be configured to cause the user device to display instructions for modifying the timing of the environmental light exposure based on the chronotype of the user, to display information associated with the ambient light exposure metric, to display information associated with the skin health metric, or a combination thereof. As noted previously herein, causing the user device to display information regarding the physiological effects may be beneficial to the user’s overall health by providing information to the user that may enable the user to understand how behavior changes (e.g., exposure to light, etc.) may help increase the user’s overall health.

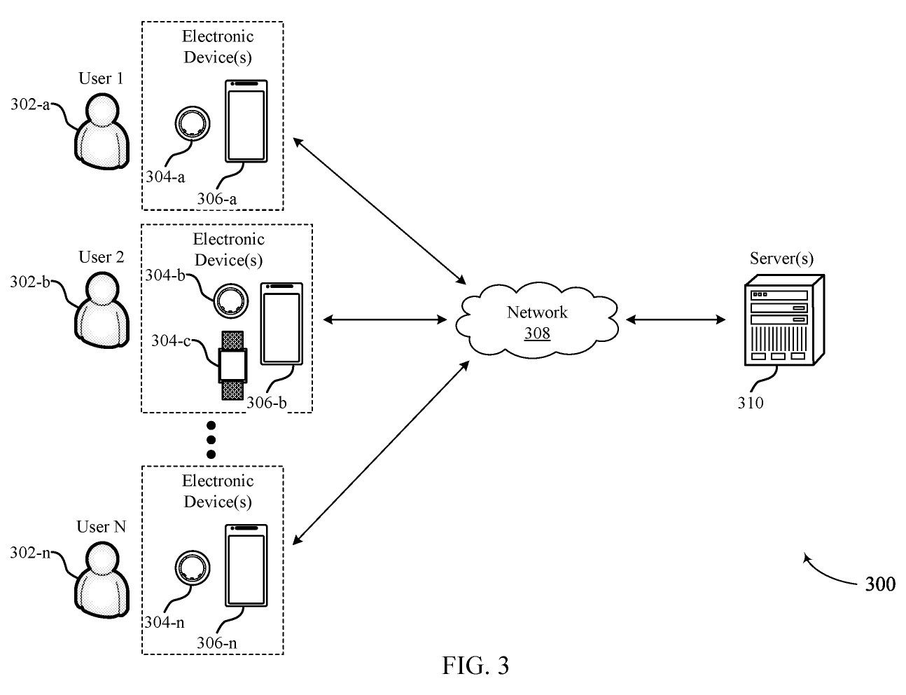

FIG. 3 illustrates an example of a system 300 that supports techniques for evaluating ambient light exposure and chronotype via wearable devices in accordance with aspects of the present disclosure. The system 300 includes a plurality of electronic devices (e.g., wearable devices 304, user devices 306) that may be worn and/or operated by one or more users 302. The system 300 further includes a network 308 and one or more servers 310.

The electronic devices may include any electronic devices known in the art, including wearable devices 304 (e.g., ring wearable devices, watch wearable devices, etc.), user devices 306 (e.g., smartphones, laptops, tablets). The electronic devices associated with the respective users 302 may include one or more of the following functionalities: 1) measuring physiological data, 2) storing the measured data, 3) processing the data, 4) providing outputs (e.g., via GUIs) to a user 302 based on the processed data, and 5) communicating data with one another and/or other computing devices. Different electronic devices may perform one or more of the functionalities.

Example wearable devices 304 may include wearable computing devices, such as a ring computing device (hereinafter “ring”) configured to be worn on a user’s 302 finger, a wrist computing device (e.g., a smart watch, fitness band, or bracelet) configured to be worn on a user’s 302 wrist, and/or a head mounted computing device (e.g., glasses/goggles). Wearable devices 304 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 304 may also be attached to, or included in, articles of clothing. For example, wearable devices 304 may be included in pockets and/or pouches on clothing. As another example, wearable device 304 may be clipped and/or pinned to clothing, or may otherwise be maintained within the vicinity of the user 302. Example articles of clothing may include, but are not limited to, hats, shirts, gloves, pants, socks, outerwear (e.g., jackets), and undergarments. In some implementations, wearable devices 304 may be included with other types of devices such as training/sporting devices that are used during physical activity. For example, wearable devices 304 may be attached to, or included in, a bicycle, skis, a tennis racket, a golf club, and/or training weights.

Much of the present disclosure may be described in the context of a wearable device 304, which may include finger-worn wearable devices, wrist-worn wearable devices, and the like. Accordingly, the terms “wearable device 304,” “wearable ring device,” “ring,” and like terms, may be used interchangeably, unless noted otherwise herein. However, the use of the terms “wearable ring device” and/or “ring” are not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 306 may include handheld mobile computing devices, such as smartphones and tablet computing devices. User devices 306 may also include personal computers, such as laptop and desktop computing devices. Other example user devices 306 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 306 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 304, user devices 306) may measure physiological parameters of respective users 302, such as photoplethysmography waveforms, continuous skin temperature, a pulse waveform, respiration rate, heart rate, heart rate variability (HRV), actigraphy, galvanic skin response, pulse oximetry, blood oxygen saturation (SpO2), blood sugar levels (e.g., glucose metrics), and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 304), mobile device application, or a server computing device may process received physiological data that was measured by other devices.

In some implementations, a user 302 may operate, or may be associated with, multiple electronic devices, some of which may measure physiological parameters and some of which may process the measured physiological parameters. In some implementations, a user 302 may have a ring (e.g., wearable device 304) that measures physiological parameters. The user 302 may also have, or be associated with, a user device 306 (e.g., mobile device, smartphone), where the wearable device 304 and the user device 306 are communicatively coupled to one another. In some cases, the user device 306 may receive data from the wearable device 304 and perform some/all of the calculations described herein. In some implementations, the user device 306 may also measure physiological parameters described herein, such as motion/activity parameters.

For example, as illustrated in FIG. 3, a first user 302-a (User 1) may operate, or may be associated with, a wearable device 304-a (e.g., wearable ring device) and a user device 306-a that may operate as described herein. In this example, the user device 306-a associated with user 302-a may process/store physiological parameters measured by the wearable device 304-a. Comparatively, a second user 302-b (User 2) may be associated with wearable devices 304-b and 304-c (e.g., wearable ring device and a wrist-worn wearable device, such as a watch) and a user device 306-b, where the user device 306-b associated with user 302-b may process/store physiological parameters measured by the wearable devices 304-b and 304-c. Moreover, an nth user 302-n (User N) may be associated with an arrangement of electronic devices described herein (e.g., wearable device 304-n, user device 306-n). In some aspects, wearable devices 304 (e.g., wearable ring devices, wrist-worn wearable devices) and other electronic devices may be communicatively coupled to the user devices 306 of the respective users 302 via Bluetooth, Wi-Fi, and other wireless protocols. Moreover, in some cases, the wearable device 304 and the user device 306 may be included within (or make up) the same device. For example, in some cases, the wearable device 304 may be configured to execute an application associated with the wearable device 304, and may be configured to display data via a GUI.

In some implementations, the wearable devices 304 (e.g., wearable ring devices) of the system 300 may be configured to collect physiological data from the respective users 302 based on arterial blood flow within the user’s finger. In particular, a wearable ring device may utilize one or more light-emitting components, such as LEDs (e.g., red LEDs, green LEDs) that emit light on the palm-side of a user’s finger to collect physiological data based on arterial blood flow within the user’s finger. In general, the terms light-emitting components, light-emitting elements, and like terms, may include, but are not limited to, LEDs, micro LEDs, mini LEDs, laser diodes (LDs) (e.g., vertical cavity surface-emitting lasers (VCSELs), and the like.

In some cases, the system 300 may be configured to collect physiological data from the respective users 302 based on blood flow diffused into a microvascular bed of skin with capillaries and arterioles. For example, the system 300 may collect PPG data based on a measured amount of blood diffused into the microvascular system of capillaries and arterioles. In some implementations, the wearable device 304 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the wearable device 304 (e.g., around an inner surface of the wearable ring device) has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a wearable ring device has been found to exhibit superior performance as compared to wearable devices worn on the wrist, as the wearable ring device may have greater access to arteries (as compared to capillaries), thereby resulting in stronger signals and more valuable physiological data.

The electronic devices of the system 300 (e.g., user devices 306, wearable devices 304) may be communicatively coupled to one or more servers 310 via wired or wireless communication protocols. For example, as shown in FIG. 3, the electronic devices (e.g., user devices 306) may be communicatively coupled to one or more servers 310 via a network 308. The network 308 may implement transfer control protocol and internet protocol (TCP/IP), such as the Internet, or may implement other network 308 protocols. Network connections between the network 308 and the respective electronic devices may facilitate transport of data via email, web, text messages, mail, or any other appropriate form of interaction within a computer network 308. For example, in some implementations, the wearable device 304-a associated with the first user 302-a may be communicatively coupled to the user device 306-a, where the user device 306-a is communicatively coupled to the servers 310 via the network 308. In additional or alternative cases, wearable devices 304 (e.g., wearable ring devices, wrist-worn wearable devices such as watches) may be directly communicatively coupled to the network 308.

The system 300 may offer an on-demand database service between the user devices 306 and the one or more servers 310. In some cases, the servers 310 may receive data from the user devices 306 via the network 308, and may store and analyze the data. Similarly, the servers 310 may provide data to the user devices 306 via the network 308. In some cases, the servers 310 may be located at one or more data centers. The servers 310 may be used for data storage, management, and processing. In some implementations, the servers 310 may provide a web-based interface to the user device 106 via web browsers.

In some aspects, the system 300 may detect periods of time that a user 302 is asleep, and classify periods of time that the user 302 is asleep into one or more sleep stages (e.g., sleep stage classification). For example, as shown in FIG. 3, User 302-a may be associated with a wearable device 304-a (e.g., wearable ring device) and a user device 306-a. In this example, the wearable device 304-a may collect physiological data associated with the user 302-a, including temperature, heart rate, HRV, respiratory rate, and the like. In some aspects, data collected by the wearable device 304-a may be input to a machine learning classifier, where the machine learning classifier is configured to determine periods of time that the user 302-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 302-a via a GUI of the user device 306-a. Sleep stage classification may be used to provide feedback to a user 302-a regarding the user’s sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 300 may utilize circadian rhythm-derived features to further improve physiological data collection, data processing procedures, and other techniques described herein. The term circadian rhythm may refer to a natural, internal process that regulates an individual’s sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 302-a via the wearable device 304-a. In this example, the circadian rhythm adjustment model may be configured to “weight,” or adjust, physiological data collected throughout a user’s natural, approximately 24-hour circadian rhythm. In some implementations, the system may initially start with a “baseline” circadian rhythm adjustment model, and may modify the baseline model using physiological data collected from each user 302 to generate tailored, individualized circadian rhythm adjustment models that are specific to each respective user 302.

In some aspects, the system 300 may utilize other biological rhythms to further improve physiological data collection, analysis, and processing by phase of these other rhythms. For example, if a weekly rhythm is detected within an individual’s baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g., in a hypothetical culture with 12 day “weeks,” 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

In some aspects, the respective devices of the system 300 may support techniques for evaluating ambient light exposure and chronotype via wearable devices. In particular, the respective components of the system 300 may be configured to measure physiological data, measure ambient light exposure, determine an effect of the ambient light exposure on the user’s physiological data, and adjusts external devices based on the effects (e.g., turn lights on/off, draw curtains/blinds, etc.). In this regard, the system 300 may be configured to selectively adjust characteristics of a user’s surrounding environment (e.g., light, curtains, blinds, etc.) based on acquired physiological data in order to improve a user’s sleep and overall health.

For example, the system 300 may perform, using one or more light-receiving components (e.g., LEDs) of a wearable device 304, one or more physiological measurements associated with physiological data of a user 302. The system 300 may perform, using the one or more light-receiving components of the wearable device 304, one or more measurements of an environmental light exposure (e.g., ambient light exposure) of the user 302. In some cases, the system 300 may determine one or more physiological effects of an environment of the user 302 on the physiological data in response to performing the one or more measurements of the environmental light exposure. In such cases, the system 300 may transmit one or more signals to cause an external device to perform one or more actions external to the wearable device 304 to adjust one or more characteristics associated with the environment of the user 302 where the one or more actions are configured to adjust the environmental light exposure of the user 302 within the environment.

In some aspects, the respective components of the system 300 may be configured to measure physiological data, measure ambient light exposure, determine an effect of the ambient light exposure on the user’s physiological data, and transmit signals to cause the user device 306 to display information associated with the effects. In this regard, the system 300 may be configured to display information based on acquired physiological data in order to improve a user’s sleep and overall health.

For example, the system 300 may perform, using one or more light-receiving components (e.g., LEDs) of a wearable device 304, one or more physiological measurements associated with physiological data of a user 302. The system 300 may perform, using the one or more light-receiving components of the wearable device 304, one or more measurements of an environmental light exposure (e.g., ambient light exposure) of the user 302. In some cases, the system 300 may determine one or more physiological effects of an environment of the user 302 on the physiological data in response to performing the one or more measurements of the environmental light exposure. In such cases, the system 300 may transmit one or more signals to cause the user device 306 associated with the wearable device 304 to display information associated with the one or more physiological effects.

It should be appreciated by a person skilled in the art that one or more aspects of the disclosure may be implemented in a system 300 to additionally, or alternatively, solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

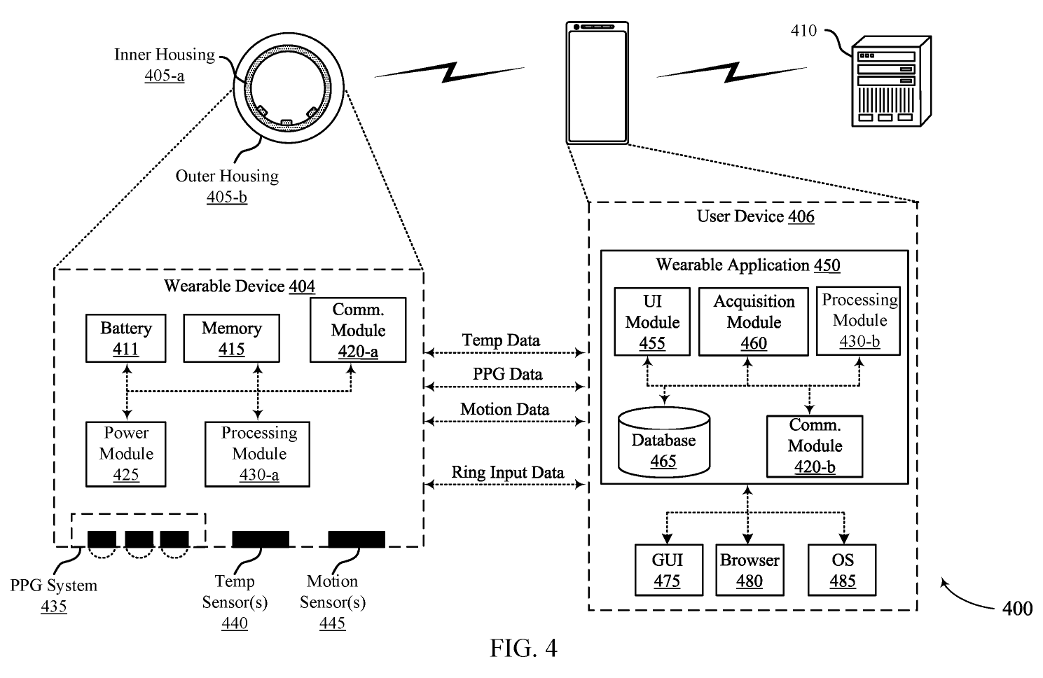

FIG. 4 illustrates an example of a system 400 that supports techniques for evaluating ambient light exposure and chronotype via wearable devices in accordance with aspects of the present disclosure. The system 400 may implement, or be implemented by, system 300. In particular, system 400 illustrates a wearable device 404 (e.g., wearable ring device), a user device 406, and a server 410, as described with reference to FIG. 3.

In some aspects, the wearable device 404 (e.g., wearable ring device) may be configured to be worn around a user’s finger, and may determine one or more user physiological parameters when worn around the user’s finger. Example measurements and determinations may include, but are not limited to, user skin temperature, pulse waveforms, respiratory rate, heart rate, HRV, blood oxygen levels (SpO2), blood sugar levels (e.g., glucose metrics), and the like.

The system 400 further includes a user device 406 (e.g., a smartphone) in communication with the wearable device 404. For example, the wearable device 404 may be in wireless and/or wired communication with the user device 406. In some implementations, the wearable device 404 may send measured and processed data (e.g., temperature data, photoplethysmogram (PPG) data, motion/accelerometer data, ring input data, and the like) to the user device 406. The user device 406 may also send data to the wearable device 404, such as firmware/configuration updates. The user device 406 may process data. In some implementations, the user device 406 may transmit data to the server 410 for processing and/or storage.

The wearable device 404 may include a housing 405 that may include an inner housing 405-a and an outer housing 405-b. In some aspects, the inner housing 405-a, the outer housing 405-b, or both, may include a curved profile/surface. In particular, the housing 405 may exhibit any curved or “circumferential” profile, including a circular profile, an elliptical profile, and the like. Moreover, in some cases, the inner housing 405-a, the outer housing 405-b, or both, may include both curved (e.g., “circumferential”) and flat/planar portions. For the purposes of the present disclosure, the term “circumferential” may be used interchangeably with the term “curved” to refer to circular-shaped, elliptical-shaped, or other curved-shaped profile.

In some aspects, the housing 405 of the wearable device 404 may store or otherwise include various components of the ring including, but not limited to, device electronics, a power source (e.g., battery 411, and/or capacitor), one or more substrates (e.g., printable circuit boards) that interconnect the device electronics and/or power source, and the like. The device electronics may include device modules (e.g., hardware/software), such as: a processing module 430-a, a memory 415, a communication module 420-a, a power module 425, and the like. The device electronics may also include one or more sensors. Example sensors may include one or more temperature sensors 440, a PPG sensor assembly (e.g., PPG system 435), and one or more motion sensors 445.

The sensors may include associated modules (not illustrated) configured to communicate with the respective components/modules of the wearable device 404, and generate signals associated with the respective sensors. In some aspects, each of the components/modules of the wearable device 404 may be communicatively coupled to one another via wired or wireless connections. Moreover, the wearable device 404 may include additional and/or alternative sensors or other components that are configured to collect physiological data from the user, including light sensors (e.g., LEDs), oximeters, and the like.

The wearable device 404 shown and described with reference to FIG. 4 is provided solely for illustrative purposes. As such, the wearable device 404 may include additional or alternative components as those illustrated in FIG. 4. Additional or alternative wearable devices 404 that provide functionality described herein may be fabricated. For example, wearable devices 404 with fewer components (e.g., sensors) may be fabricated. In a specific example, a wearable device 404 with a single temperature sensor 440 (or other sensor), a power source, and device electronics configured to read the single temperature sensor 440 (or other sensor) may be fabricated. In another specific example, a temperature sensor 440 (or other sensor) may be attached to a user’s finger (e.g., using adhesives, wraps, clamps, spring loaded clamps, etc.). In this case, the sensor may be wired to another computing device, such as a wrist worn computing device that reads the temperature sensor 440 (or other sensor). In other examples, a wearable device 404 that includes additional sensors and processing functionality may be fabricated.

The housing 405 may include one or more housing components. The housing 405 may include an outer housing 405-b component (e.g., a shell) and an inner housing 405-a component (e.g., a molding). The housing 405 may include additional components (e.g., additional layers) not explicitly illustrated in FIG. 4. For example, in some implementations, the wearable device 404 may include one or more insulating layers that electrically insulate the device electronics and other conductive materials (e.g., electrical traces) from the outer housing 405-b. The housing 405 may provide structural support for the device electronics, battery 411, substrate(s), and other components. For example, the housing 405 may protect the device electronics, battery 411, and substrate(s) from mechanical forces, such as pressure and impacts. The housing 405 may also protect the device electronics, battery 411, and substrate(s) from water and/or other chemicals.

The inner housing 405-a may be configured to interface with the user’s finger. The inner housing 405-a may be formed from a polymer (e.g., a medical grade polymer) or other material. In some implementations, the inner housing 405-a may be transparent. For example, the inner housing 405-a may be transparent to light emitted by the PPG LEDs. In some implementations, the inner housing 405-a component may be molded onto the outer housing 405-b. For example, the inner housing 405-a may include a polymer that is molded (e.g., injection molded) to fit into an outer housing 405-b metallic shell.

The inner housing 405-a and the outer housing 405-b may be fabricated from one or more materials. In some implementations, the inner housing 405-a, the outer housing 405-b, or both, may include a metal, such as titanium, that may provide strength and abrasion resistance at a relatively light weight. Additionally, or alternatively, the inner housing 405-a, and/or the outer housing 405-b may also be fabricated from other materials, such polymers, plastic materials, epoxy materials, ceramic materials, and the like. In some implementations, the outer housing 405-b may be protective as well as decorative.

The wearable device 404 may include one or more substrates (not illustrated). The device electronics and battery 411 may be included on the one or more substrates. For example, the device electronics and battery 411 may be mounted on one or more substrates. Example substrates may include one or more printed circuit boards (PCBs), such as flexible PCB (e.g., polyimide). In some implementations, the electronics/battery 411 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 411 to the device electronics.

The device electronics, battery 411, and substrates may be arranged in the wearable device 404 in a variety of ways. In some implementations, one substrate that includes device electronics may be mounted along the bottom of the wearable device 404 (e.g., the bottom half), such that the sensors (e.g., PPG system 435, temperature sensors 440, motion sensors 445, and other sensors) interface with the underside of the user’s finger. In these implementations, the battery 411 may be included along the top portion of the wearable device 404 (e.g., on another substrate).

The various components/modules of the wearable device 404 represent functionality (e.g., circuits and other components) that may be included in the wearable device 404. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 415 (memory module) of the wearable device 404 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 415 may store any of the data described herein. For example, the memory 415 may be configured to store data (e.g., motion data, temperature data, PPG data) collected by the respective sensors and PPG system 435. Furthermore, memory 415 may include instructions that, when executed by one or more processing circuits, cause the modules to perform various functions attributed to the modules herein. The device electronics of the wearable device 404 described herein are only example device electronics. As such, the types of electronic components used to implement the device electronics may vary based on design considerations.

The functions attributed to the modules of the wearable device 404 (e.g., wearable ring device) described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 430-a of the wearable device 404 may include one or more processors (e.g., processing units), microcontrollers, digital signal processors, systems on a chip (SOCs), and/or other processing devices. The processing module 430-a communicates with the modules included in the wearable device 404. For example, the processing module 430-a may transmit/receive data to/from the modules and other components of the wearable device 404, such as the sensors. As described herein, the modules may be implemented by various circuit components. Accordingly, the modules may also be referred to as circuits (e.g., a communication circuit and power circuit).

The processing module 430-a may communicate with the memory 415. The memory 415 may include computer-readable instructions that, when executed by the processing module 430-a, cause the processing module 430-a to perform the various functions attributed to the processing module 430-a herein. In some implementations, the processing module 430-a (e.g., a microcontroller) may include additional features associated with other modules, such as communication functionality provided by the communication module 420-a (e.g., an integrated Bluetooth Low Energy transceiver) and/or additional onboard memory 415.

The communication module 420-a may include circuits that provide wireless and/or wired communication with the user device 406 (e.g., communication module 420-b of the user device 406). In some implementations, the communication modules 420-a, 420-b may include wireless communication circuits, such as Bluetooth circuits and/or Wi-Fi circuits. In some implementations, the communication modules 420-a, 420-b can include wired communication circuits, such as Universal Serial Bus (USB) communication circuits. Using the communication module 420-a, the wearable device 404 and the user device 406 may be configured to communicate with each other. The processing module 430-a of the ring may be configured to transmit/receive data to/from the user device 406 via the communication module 420-a. Example data may include, but is not limited to, motion data, temperature data, pulse waveforms, heart rate data, HRV data, PPG data, and status updates (e.g., charging status, battery charge level, and/or wearable device 404 configuration settings). The processing module 430-a of the ring may also be configured to receive updates (e.g., software/firmware updates) and data from the user device 406.

The wearable device 404 may include a battery 411 (e.g., a rechargeable battery 411). An example battery 411 may include a Lithium-Ion or Lithium-Polymer type battery 411, although a variety of battery 411 options are possible. The battery 411 may be wirelessly charged. In some implementations, the wearable device 404 may include a power source other than the battery 411, such as a capacitor. The power source (e.g., battery 411 or capacitor) may have a curved geometry that matches the curve of the wearable device 404. In some aspects, a charger or other power source may include additional sensors that may be used to collect data in addition to, or that supplements, data collected by the wearable device 404 itself. Moreover, a charger or other power source for the wearable device 404 may function as a user device 406, in which case the charger or other power source for the wearable device 404 may be configured to receive data from the wearable device 404, store and/or process data received from the wearable device 404, and communicate data between the wearable device 404 and the servers 410.

In some aspects, the wearable device 404 includes a power module 425 that may control charging of the battery 411. For example, the power module 425 may interface with an external wireless charger that charges the battery 411 when interfaced with the wearable device 404. The charger may include a datum structure that mates with a wearable device 404 datum structure to create a specified orientation with the wearable device 404 during charging. The power module 425 may also regulate voltage(s) of the device electronics, regulate power output to the device electronics, and monitor the state of charge of the battery 411. In some implementations, the battery 411 may include a protection circuit module (PCM) that protects the battery 411 from high current discharge, over voltage during charging, and under voltage during discharge. The power module 425 may also include electro-static discharge (ESD) protection.

The one or more temperature sensors 440 may be electrically coupled to the processing module 430-a. The temperature sensor 440 may be configured to generate a temperature signal (e.g., temperature data) that indicates a temperature read or sensed by the temperature sensor 440. The processing module 430-a may determine a temperature of the user in the location of the temperature sensor 440. For example, in the wearable device 404, temperature data generated by the temperature sensor 440 may indicate a temperature of a user at the user’s finger (e.g., skin temperature). In some implementations, the temperature sensor 440 may contact the user’s skin. In other implementations, a portion of the housing 405 (e.g., the inner housing 405-a) may form a barrier (e.g., a thin, thermally conductive barrier) between the temperature sensor 440 and the user’s skin. In some implementations, portions of the wearable device 404 configured to contact the user’s finger may have thermally conductive portions and thermally insulative portions. The thermally conductive portions may conduct heat from the user’s finger to the temperature sensors 440. The thermally insulative portions may insulate portions of the wearable device 404 (e.g., the temperature sensor 440) from ambient temperature.

In some implementations, the temperature sensor 440 may generate a digital signal (e.g., temperature data) that the processing module 430-a may use to determine the temperature. As another example, in cases where the temperature sensor 440 includes a passive sensor, the processing module 430-a (or a temperature sensor 440 module) may measure a current/voltage generated by the temperature sensor 440 and determine the temperature based on the measured current/voltage. Example temperature sensors 440 may include a thermistor, such as a negative temperature coefficient (NTC) thermistor, or other types of sensors including resistors, transistors, diodes, and/or other electrical/electronic components.

The processing module 430-a may sample the user’s temperature over time. For example, the processing module 430-a may sample the user’s temperature according to a sampling rate. An example sampling rate may include one sample per second, although the processing module 430-a may be configured to sample the temperature signal at other sampling rates that are higher or lower than one sample per second. In some implementations, the processing module 430-a may sample the user’s temperature continuously throughout the day and night. Sampling at a sufficient rate (e.g., one sample per second) throughout the day may provide sufficient temperature data for analysis described herein.

The processing module 430-a may store the sampled temperature data in memory 415. In some implementations, the processing module 430-a may process the sampled temperature data. For example, the processing module 430-a may determine average temperature values over a period of time. In one example, the processing module 430-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 415 may store the average temperature values over time. In some implementations, the memory 415 may store average temperatures (e.g., one per minute) instead of sampled temperatures in order to conserve memory 415.

The sampling rate, which may be stored in memory 415, may be configurable. In some implementations, the sampling rate may be the same throughout the day and night. In other implementations, the sampling rate may be changed throughout the day/night. In some implementations, the wearable device 404 may filter/reject temperature readings, such as large spikes in temperature that are not indicative of physiological changes (e.g., a temperature spike from a hot shower). In some implementations, the wearable device 404 may filter/reject temperature readings that may not be reliable due to other factors, such as excessive motion during exercise (e.g., as indicated by a motion sensor 445).

The wearable device 404 (e.g., communication module) may transmit the sampled and/or average temperature data to the user device 406 for storage and/or further processing. The user device 406 may transfer the sampled and/or average temperature data to the server 410 for storage and/or further processing.

Although the wearable device 404 is illustrated as including a single temperature sensor 440, the wearable device 404 may include multiple temperature sensors 440 in one or more locations, such as arranged along the inner housing 405-a near the user’s finger. In some implementations, the temperature sensors 440 may be stand-alone temperature sensors 440. Additionally, or alternatively, one or more temperature sensors 440 may be included with other components (e.g., packaged with other components), such as with the accelerometer and/or processor.

The processing module 430-a may acquire and process data from multiple temperature sensors 440 in a similar manner described with respect to a single temperature sensor 440. For example, the processing module 430 may individually sample, average, and store temperature data from each of the multiple temperature sensors 440. In other examples, the processing module 430-a may sample the sensors at different rates and average/store different values for the different sensors. In some implementations, the processing module 430-a may be configured to determine a single temperature based on the average of two or more temperatures determined by two or more temperature sensors 440 in different locations on the finger.

The temperature sensors 440 on the wearable device 404 (e.g., wearable ring device) may acquire distal temperatures at the user’s finger (e.g., any finger). For example, one or more temperature sensors 440 on the wearable device 404 may acquire a user’s temperature from the underside of a finger or at a different location on the finger. In some implementations, the wearable device 404 may continuously acquire distal temperature (e.g., at a sampling rate). Although distal temperature measured by a wearable device 404 at the finger is described herein, other devices may measure temperature at the same/different locations. In some cases, the distal temperature measured at a user’s finger may differ from the temperature measured at a user’s wrist or other external body location. Additionally, the distal temperature measured at a user’s finger (e.g., a “shell” temperature) may differ from the user’s core temperature. As such, the wearable device 404 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The wearable device 404 may include a PPG system 435. The PPG system 435 may include one or more optical transmitters that transmit light. The PPG system 435 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user’s finger. The PPG signal generated by the PPG system 435 may indicate the perfusion of blood in the illuminated region. For example, the PPG signal may indicate blood volume changes in the illuminated region caused by a user’s pulse pressure. The processing module 430-a may sample the PPG signal and determine a user’s pulse waveform based on the PPG signal. The processing module 430-a may determine a variety of physiological parameters based on the user’s pulse waveform, such as a user’s respiratory rate, heart rate, HRV, oxygen saturation, and other circulatory parameters.

In some implementations, the PPG system 435 may be configured as a reflective PPG system 435 where the optical receiver(s) receive transmitted light that is reflected through the region of the user’s finger. In some implementations, the PPG system 435 may be configured as a transmissive PPG system 435 where the optical transmitter(s) and optical receiver(s) are arranged opposite to one another, such that light is transmitted directly through a portion of the user’s finger to the optical receiver(s).

The number and ratio of transmitters and receivers included in the PPG system 435 may vary. Example optical transmitters may include LEDs. The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 435.

The PPG system 435 illustrated in FIG. 4 may include a reflective PPG system 435 in some implementations. In these implementations, the PPG system 435 may include a centrally located optical receiver (e.g., at the bottom of the wearable device 404) and two optical transmitters located on each side of the optical receiver. In this implementation, the PPG system 435 (e.g., optical receiver) may generate the PPG signal based on light received from one or both of the optical transmitters. In other implementations, other placements, combinations, and/or configurations of one or more optical transmitters and/or optical receivers are contemplated.

The processing module 430-a may control one or both of the optical transmitters to transmit light while sampling the PPG signal generated by the optical receiver. In some implementations, the processing module 430-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 435 may result in a pulse waveform that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 430-a may store the pulse waveform in memory 415 in some implementations. The processing module 430-a may process the pulse waveform as it is generated and/or from memory 415 to determine user physiological parameters described herein.

The processing module 430-a may determine the user’s heart rate based on the pulse waveform. For example, the processing module 430-a may determine heart rate (e.g., in beats per minute) based on the time between peaks in the pulse waveform. The time between peaks may be referred to as an interbeat interval (IBI). The processing module 430-a may store the determined heart rate values and IBI values in memory 415.

The processing module 430-a may determine HRV over time. For example, the processing module 430-a may determine HRV based on the variation in the IBIs. The processing module 430-a may store the HRV values over time in the memory 415. Moreover, the processing module 430-a may determine the user’s respiratory rate over time. For example, the processing module 430-a may determine respiratory rate based on frequency modulation, amplitude modulation, or baseline modulation of the user’s IBI values over a period of time. Respiratory rate may be calculated in breaths per minute or as another breathing rate (e.g., breaths per 30 seconds). The processing module 430-a may store user respiratory rate values over time in the memory 415.

The wearable device 404 may include one or more motion sensors 445, such as one or more accelerometers (e.g., 6-D accelerometers) and/or one or more gyroscopes (gyros). The motion sensors 445 may generate motion signals that indicate motion of the sensors. For example, the wearable device 404 may include one or more accelerometers that generate acceleration signals that indicate acceleration of the accelerometers. As another example, the wearable device 404 may include one or more gyro sensors that generate gyro signals that indicate angular motion (e.g., angular velocity) and/or changes in orientation. The motion sensors 445 may be included in one or more sensor packages. An example accelerometer/gyro sensor is a Bosch BMI160 inertial micro electro-mechanical system (MEMS) sensor that may measure angular rates and accelerations in three perpendicular axes.

The processing module 430-a may sample the motion signals at a sampling rate (e.g., 50Hz) and determine the motion of the wearable device 404 based on the sampled motion signals. For example, the processing module 430-a may sample acceleration signals to determine acceleration of the wearable device 404. As another example, the processing module 430-a may sample a gyro signal to determine angular motion. In some implementations, the processing module 430-a may store motion data in memory 415. Motion data may include sampled motion data as well as motion data that is calculated based on the sampled motion signals (e.g., acceleration and angular values).

The wearable device 404 may store a variety of data described herein. For example, the wearable device 404 may store temperature data, such as raw sampled temperature data and calculated temperature data (e.g., average temperatures). As another example, wearable device 404 may store PPG signal data, such as pulse waveforms and data calculated based on the pulse waveforms (e.g., heart rate values, IBI values, HRV values, and respiratory rate values). The wearable device 404 may also store motion data, such as sampled motion data that indicates linear and angular motion.

The wearable device 404, or other computing device, may calculate and store additional values based on the sampled/calculated physiological data. For example, the processing module 430 may calculate and store various metrics, such as sleep metrics (e.g., a Sleep Score), activity metrics, and readiness metrics. In some implementations, additional values/metrics may be referred to as “derived values.” The wearable device 404, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the wearable device 404 is oriented on the user’s finger and if the wearable device 404 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 430-a may compress the data stored in memory 415. For example, the processing module 430-a may delete sampled data after making calculations based on the sampled data. As another example, the processing module 430-a may average data over longer periods of time in order to reduce the number of stored values. In a specific example, if average temperatures for a user over one minute are stored in memory 415, the processing module 430-a may calculate average temperatures over a five minute time period for storage, and then subsequently erase the one minute average temperature data. The processing module 430-a may compress data based on a variety of factors, such as the total amount of used/available memory 415 and/or an elapsed time since the wearable device 404 last transmitted the data to the user device 406.

Although a user’s physiological parameters may be measured by sensors included on a wearable device 404, other devices may measure a user’s physiological parameters. For example, although a user’s temperature may be measured by a temperature sensor 440 included in a wearable device 404, other devices may measure a user’s temperature. In some examples, other wearable devices (e.g., wrist devices) may include sensors that measure user physiological parameters. Additionally, medical devices, such as external medical devices (e.g., wearable medical devices) and/or implantable medical devices, may measure a user’s physiological parameters. One or more sensors on any type of computing device may be used to implement the techniques described herein.

The physiological measurements may be taken continuously throughout the day and/or night. In some implementations, the physiological measurements may be taken during portions of the day and/or portions of the night. In some implementations, the physiological measurements may be taken in response to determining that the user is in a specific state, such as an active state, resting state, and/or a sleeping state. For example, the wearable device 404 can make physiological measurements in a resting/sleep state in order to acquire cleaner physiological signals. In one example, the wearable device 404 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the wearable device 404 may be configured to collect, store, and/or process data, and may transfer any of the data described herein to the user device 406 for storage and/or processing. In some aspects, the user device 406 includes a wearable application 450, an operating system 485 (OS), a web browser application (e.g., web browser 480), one or more additional applications, and a GUI 475. The user device 406 may further include other modules and components, including sensors, audio devices, haptic feedback devices, and the like. The wearable application 450 may include an example of an application (e.g., “app”) that may be installed on the user device 406. The wearable application 450 may be configured to acquire data from the wearable device 404, store the acquired data, and process the acquired data as described herein. For example, the wearable application 450 may include a user interface (UI) module 455, an acquisition module 460, a processing module 430-b, a communication module 420-b, and a storage module (e.g., database 465) configured to store application data.

In some cases, the wearable device 404 and the user device 406 may be included within (or make up) the same device. For example, in some cases, the wearable device 404 may be configured to execute the wearable application 450, and may be configured to display data via the GUI 475.

The various data processing operations described herein may be performed by the wearable device 404, the user device 406, the servers 410, or any combination thereof. For example, in some cases, data collected by the wearable device 404 may be pre-processed and transmitted to the user device 406. In this example, the user device 406 may perform some data processing operations on the received data, may transmit the data to the servers 410 for data processing, or both. For instance, in some cases, the user device 406 may perform processing operations that require relatively low processing power and/or operations that require a relatively low latency, whereas the user device 406 may transmit the data to the servers 410 for processing operations that require relatively high processing power and/or operations that may allow relatively higher latency.

In some aspects, data collected by the wearable device 404, and/or analyses performed by the wearable device 404, the user device 406, and/or the servers 410, may be used to adjust operational parameters of the wearable device 404. For example, based on a determined heart rate of the user and/or a determined activity state of the user (and/or based on other determined parameters or characteristics, such as measured ambient light levels or other environmental characteristics), the wearable device 404 may adjust a sampling rate for measuring the user’s heart rate, and/or may activate or deactivate certain sensors and/or physiological measurements (e.g., deactivate SpO2 measurements when the user is engaged in physical activity, or otherwise exhibits an activity/movement level above some threshold). By way of another example, the user device 406 and/or the servers 410 may calculate a Readiness Score for the user, and may deactivate or disable activity measurements performed by the wearable device 404 in cases where the Readiness Score is below some threshold (in order to reduce power consumption and conserve battery at the wearable device 404, and/or to disincentivize the user from performing rigorous activity when their Readiness Score is below the threshold value). In this regard, any measurements, calculations, and/or analyses performed by the various devices within the system 400 (e.g., wearable device 404, user device 406, servers 410) may be used by the system 400 to control and/or adjust the operational parameters of the wearable device 404.

Operational parameters that may be controlled/adjusted at the wearable device 404 (and/or other external devices)based on collected data and/or analyses performed by the system 400 may include, but are not limited to, a periodicity/frequency that measurements are performed (e.g., sampling rate), a power level or intensity of LEDs, algorithms used to analyze data at the wearable device 404, what types of measurements are performed (e.g., enabling/disabling specific sensors or types of measurements), a periodicity or frequency that the wearable device 404 transmits data to the user device 406, or any combination thereof. Adjusting operational parameters of the wearable device 404 based on collected data and/or analyses performed by the system 400 may reduce power consumption and improve battery performance at the wearable device 404, and may lead to higher quality data collected by the wearable device 404, thereby enabling the system 400 to perform more accurate and reliable analyses/diagnoses of the user’s physiological parameters, and leading to better guidance and insights that may enable the user to improve their overall health.

In some aspects, the wearable device 404 (e.g., wearable ring device), user device 406, and server 410 of the system 400 may be configured to evaluate sleep patterns for a user. In particular, the respective components of the system 400 may be used to collect data from a user via the wearable device 404, and generate one or more scores (e.g., Sleep Score, Readiness Score) for the user based on the collected data. For example, as noted previously herein, the wearable device 404 of the system 400 may be worn by a user to collect data from the user, including temperature, heart rate, HRV, and the like. Data collected by the wearable device 404 may be used to determine when the user is asleep in order to evaluate the user’s sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the wearable device 404 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 400 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user’s overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user’s sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user’s overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user’s needs. Typically, adults need 7–9 hours of sleep a night to stay healthy, alert, and to perform at their best both mentally and physically. However, it is normal to have an occasional night of bad sleep, so the sleep balance contributor takes into account long-term sleep patterns to determine whether each user’s sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user’s resting heart rate to stabilize during the night. A sign of a very good recovery is that the user’s resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user’s highest temperature during the nap is at least 0.5°C higher than the highest temperature during the longest period. In some aspects, the ring may measure a user’s body temperature while the user is asleep, and the system 400 may display the user’s average temperature relative to the user’s baseline temperature. If a user’s body temperature is outside of their normal range (e.g., clearly above or below 0.0), the body temperature contributor may be highlighted (e.g., go to a “Pay attention” state) or otherwise generate an alert for the user.

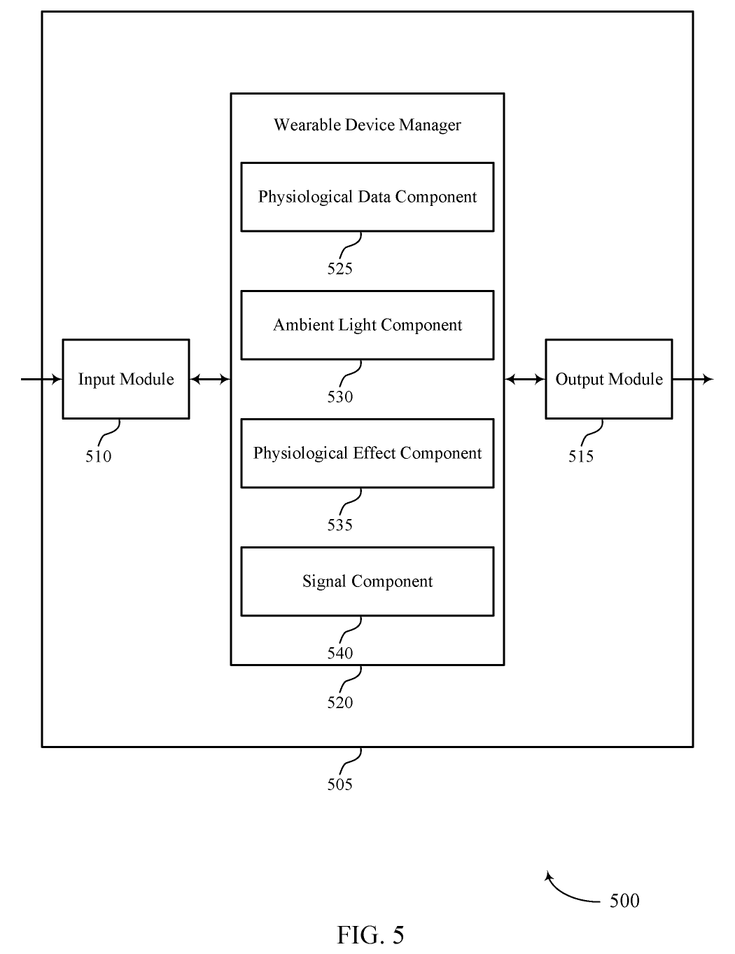

FIG. 5 shows a block diagram 500 of a device 505 that supports techniques for evaluating ambient light exposure and chronotype via wearable devices in accordance with aspects of the present disclosure. The device 505 may include an input module 510, an output module 515, and a wearable device manage 520. The device 505, or one or more components of the device 505 (e.g., the input module 510, the output module 515, the wearable device manager 520), may include at least one processor, which may be coupled with at least one memory, to support the described techniques. Each of these components may be in communication with one another (e.g., via one or more buses).

The input module 510 may provide a means for receiving information such as packets, user data, control information, or any combination thereof associated with various information channels (e.g., control channels, data channels, information channels related to illness detection techniques). Information may be passed on to other components of the device 505. The input module 510 may utilize a single antenna or a set of multiple antennas.

The output module 515 may provide a means for transmitting signals generated by other components of the device 505. For example, the output module 515 may transmit information such as packets, user data, control information, or any combination thereof associated with various information channels (e.g., control channels, data channels, information channels related to illness detection techniques). In some examples, the output module 515 may be co-located with the input module 510 in a transceiver module. The output module 515 may utilize a single antenna or a set of multiple antennas.

For example, the wearable device manager 520 may include a physiological data component 525, an ambient light component 530, a physiological effect component 535, a signal component 540, or any combination thereof. In some examples, the wearable device manager 520, or various components thereof, may be configured to perform various operations (e.g., receiving, monitoring, transmitting) using or otherwise in cooperation with the input module 510, the output module 515, or both. For example, the wearable device manager 520 may receive information from the input module 510, send information to the output module 515, or be integrated in combination with the input module 510, the output module 515, or both to receive information, transmit information, or perform various other operations as described herein.

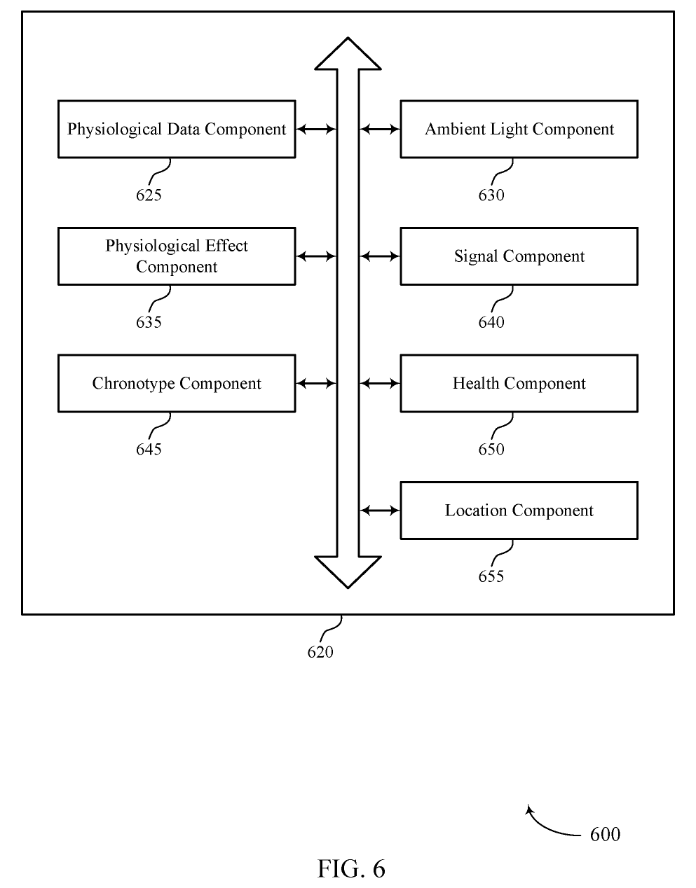

The physiological data component 525 may be configured as or otherwise support a means for performing, using one or more light-receiving components of a wearable device, one or more physiological measurements associated with physiological data of a user. The ambient light component 530 may be configured as or otherwise support a means for performing, using the one or more light-receiving components of the wearable device, one or more measurements of an environmental light exposure of the user while the wearable device is worn by the user, the environmental light exposure representing ambient light incident on the wearable device that it not attributable to light emitted by the wearable device. The physiological effect component 535 may be configured as or otherwise support a means for determining one or more physiological effects of an environment of the user on the physiological data based at least in part on performing the one or more measurements of the environmental light exposure. The signal component 540 may be configured as or otherwise support a means for generating an alert or instruction to modify the environmental light exposure; or transmitting a control signal to at least one external device to adjust the environment of the user in a manner that modifies the environmental light exposure of the user. FIG. 6 shows a block diagram 600 of a wearable device manager 620 that supports techniques for evaluating ambient light exposure and chronotype via wearable devices in accordance with aspects of the present disclosure. The wearable device manager 620 may be an example of aspects of a wearable device manager. The wearable device manager 620, or various components thereof, may be an example of means for performing various aspects of techniques for evaluating ambient light exposure and chronotype via wearable devices as described herein. For example, the wearable device manager 620 may include a physiological data component 625, an ambient light component 630, a physiological effect component 635, a signal component 640, a chronotype component 645, a health component 650, a location component 655, or any combination thereof. Each of these components, or components of subcomponents thereof (e.g., one or more processors, one or more memories), may communicate, directly or indirectly, with one another (e.g., via one or more buses).

The physiological data component 625 may be configured as or otherwise support a means for performing, using one or more light-receiving components of a wearable device, one or more physiological measurements associated with physiological data of a user. The ambient light component 630 may be configured as or otherwise support a means for performing, using the one or more light-receiving components of the wearable device, one or more measurements of an environmental light exposure of the user while the wearable device is worn by the user, the environmental light exposure representing ambient light incident on the wearable device that it not attributable to light emitted by the wearable device. The physiological effect component 635 may be configured as or otherwise support a means for determining one or more physiological effects of an environment of the user on the physiological data based at least in part on performing the one or more measurements of the environmental light exposure. The signal component 640 may be configured as or otherwise support a means for generating an alert or instruction to modify the environmental light exposure; or transmitting a control signal to at least one external device to adjust the environment of the user in a manner that modifies the environmental light exposure of the user.

In some examples, the chronotype component 645 may be configured as or otherwise support a means for determining a chronotype of the user based at least in part on the one or more physiological measurements. In some examples, the chronotype component 645 may be configured as or otherwise support a means for determining a timing of the environmental light exposure relative to the chronotype based at least in part on the one or more measurements of the environmental light exposure, wherein the one or more physiological effects of the environment are based at least in part on the timing of the environmental light exposure relative to the chronotype.

In some examples, the signal component 640 may be configured as or otherwise support a means for transmitting one or more signals to a user device associated with the wearable device to cause the user device to display instructions for modifying the timing of the environmental light exposure based at least in part on the chronotype of the user.

In some examples, the ambient light component 630 may be configured as or otherwise support a means for calculating an ambient light exposure metric of the user based at least in part on the one or more measurements of the environmental light exposure. In some examples, the signal component 640 may be configured as or otherwise support a means for transmitting one or more signals to a user device associated with the wearable device to cause the user device to display information associated with the ambient light exposure metric.

In some examples, the health component 650 may be configured as or otherwise support a means for calculating a skin health metric of the user based at least in part on the one or more measurements of the environmental light exposure. In some examples, the signal component 640 may be configured as or otherwise support a means for transmitting one or more signals to a user device associated with the wearable device to cause the user device to display information associated with the skin health metric.

In some examples, the location component 655 may be configured as or otherwise support a means for receiving geographical location data associated with a location of the user during a time interval of the one or more measurements of an environmental light exposure. In some examples, the ambient light component 630 may be configured as or otherwise support a means for determining an ultraviolet light exposure of the user based at least in part on the geographical location data and the one or more measurements of an environmental light exposure, wherein the skin health metric is based at least in part on the ultraviolet light exposure.

In some examples, the ambient light component 630 may be configured as or otherwise support a means for determining at least a first wavelength exposure and a second wavelength exposure of the user based at least in part on the one or more measurements of the environmental light exposure, wherein the one or more physiological effects are based at least in part on the first wavelength exposure, the second wavelength exposure, or both.

In some examples, the location component 655 may be configured as or otherwise support a means for determining a location of the user for at least a portion of a time interval associated with the one or more measurements of the environmental light exposure based at least in part on the first wavelength exposure, the second wavelength exposure, or both, wherein the location comprises an indoor location or an outdoor location.

In some examples, the one or more physiological measurements are performed using the one or more light-receiving components and one or more light-transmitting components of the wearable device during a first set of measurement occasions at the wearable device. In some examples, the one or more measurements of the environmental light exposure are performed during a second set of measurement occasions that are non-overlapping in time with respect to the first set of measurement occasions.

In some examples, the physiological effect component 635 may be configured as or otherwise support a means for receiving, via a user device associated with the wearable device, a user input indicating one or more relationships for using the at least one external device to modify the environmental light exposure based on the one or more physiological effects, wherein transmitting the control signal is based at least in part on the user input indicating the one or more relationships.

In some examples, the health component 650 may be configured as or otherwise support a means for calculating a dosage of vitamin D for the user based at least in part on the one or more measurements of an environmental light exposure. In some examples, the health component 650 may be configured as or otherwise support a means for instructing, via a user device associated with the wearable device, the user to intake the dosage of vitamin D.

In some examples, the at least one external device comprises an external light source, curtains, shades, blinds, or any combination thereof.

In some examples, the wearable device comprises a wearable ring device. In some examples, the one or more light-receiving components are positioned on an inner curved surface of the wearable ring device. In some examples, the inner curved surface is configured to at least partially contact a tissue of the user when the wearable ring device is worn by the user.

In some examples, the wearable device comprises a wearable ring device. In some examples, the one or more light-receiving components are positioned on an inner curved surface of the wearable ring device. In some examples, the inner curved surface is configured to at least partially contact a tissue of the user when the wearable ring device is worn by the user.

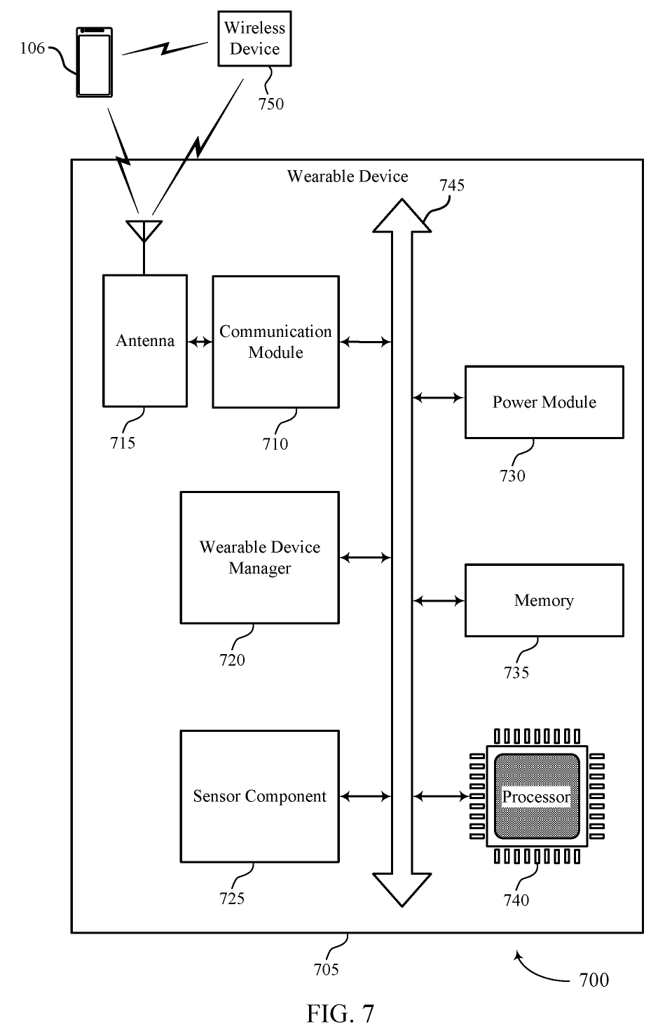

FIG. 7 shows a diagram of a system 700 including a device 705 that supports techniques for evaluating ambient light exposure and chronotype via wearable devices in accordance with aspects of the present disclosure. The device 705 may include an example of a wearable device 104, as described previously herein. The device 705 may include components for bi-directional communications including components for transmitting and receiving communications with a user device 406 and a server 410, such as a wearable device manager 720, a communication module 710, one or more antennas 715, a sensor component 725, a power module 730, at least one memory 735, at least one processor 740, and a wireless device 750. These components may be in electronic communication or otherwise coupled (e.g., operatively, communicatively, functionally, electronically, electrically) via one or more buses (e.g., a bus 745).

For example, the wearable device manager 720 may be configured as or otherwise support a means for performing, using one or more light-receiving components of a wearable device, one or more physiological measurements associated with physiological data of a user. The wearable device manager 720 may be configured as or otherwise support a means for performing, using the one or more light-receiving components of the wearable device, one or more measurements of an environmental light exposure of the user while the wearable device is worn by the user, the environmental light exposure representing ambient light incident on the wearable device that it not attributable to light emitted by the wearable device. The wearable device manager 720 may be configured as or otherwise support a means for determining one or more physiological effects of an environment of the user on the physiological data based at least in part on performing the one or more measurements of the environmental light exposure. The wearable device manager 720 may be configured as or otherwise support a means for generating an alert or instruction to modify the environmental light exposure; or transmitting a control signal to at least one external device to adjust the environment of the user in a manner that modifies the environmental light exposure of the user.

By including or configuring the wearable device manager 720 in accordance with examples as described herein, the device 705 may support techniques for improved communication reliability, reduced latency, improved user experience related to reduced processing, reduced power consumption, more efficient utilization of communication resources, improved coordination between devices, longer battery life, and improved utilization of processing capability.

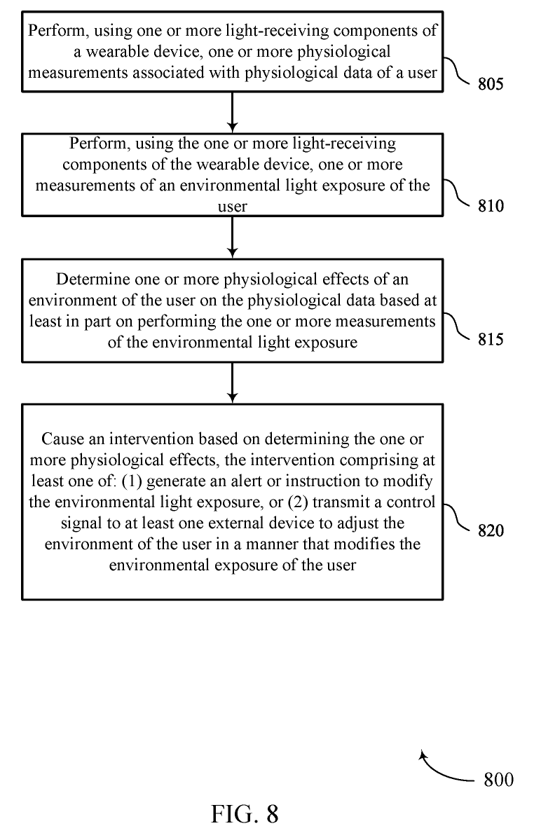

FIG. 8 shows a flowchart illustrating a method 800 that supports techniques for evaluating ambient light exposure and chronotype via wearable devices in accordance with aspects of the present disclosure. The operations of the method 800 may be implemented by a wearable device or its components as described herein. For example, the operations of the method 800 may be performed by a wearable device as described with reference to FIGS. 3 through 7. In some examples, a wearable device may execute a set of instructions to control the functional elements of the wearable device to perform the described functions. Additionally, or alternatively, the wearable device may perform aspects of the described functions using special-purpose hardware.

At 805, the method may include performing, using one or more light-receiving components of a wearable device, one or more physiological measurements associated with physiological data of a user. The operations of 805 may be performed in accordance with examples as disclosed herein. In some examples, aspects of the operations of 805 may be performed by a physiological data component 525 as described with reference to FIG. 5.

At 810, the method may include performing, using the one or more light-receiving components of the wearable device, one or more measurements of an environmental light exposure of the user while the wearable device is worn by the user, the environmental light exposure representing ambient light incident on the wearable device that is not attributable to light emitted by the wearable device. The operations of 810 may be performed in accordance with examples as disclosed herein. In some examples, aspects of the operations of 810 may be performed by an ambient light component 530 as described with reference to FIG. 5.

At 815, the method may include determining one or more physiological effects of an environment of the user on the physiological data based at least in part on performing the one or more measurements of the environmental light exposure. The operations of 815 may be performed in accordance with examples as disclosed herein. In some examples, aspects of the operations of 815 may be performed by a physiological effect component 535 as described with reference to FIG. 5.

At 820, the method may include causing an intervention based on determining the one or more physiological effects, the intervention comprising at least one of: (1) generating an alert or instruction to modify the environmental light exposure, or (2) transmitting a control signal to at least one external device to adjust the environment of the user in a manner that modifies the environmental exposure of the user. This approach may automatically adjusts the environmental light exposure in the environment of the user to alter the one or more physiological effects on the user, which provides improved management of the physiological status of the user. The operations of 820 may be performed in accordance with examples as disclosed herein. In some examples, aspects of the operations of 820 may be performed by a signal component 540 as described with reference to FIG. 5.

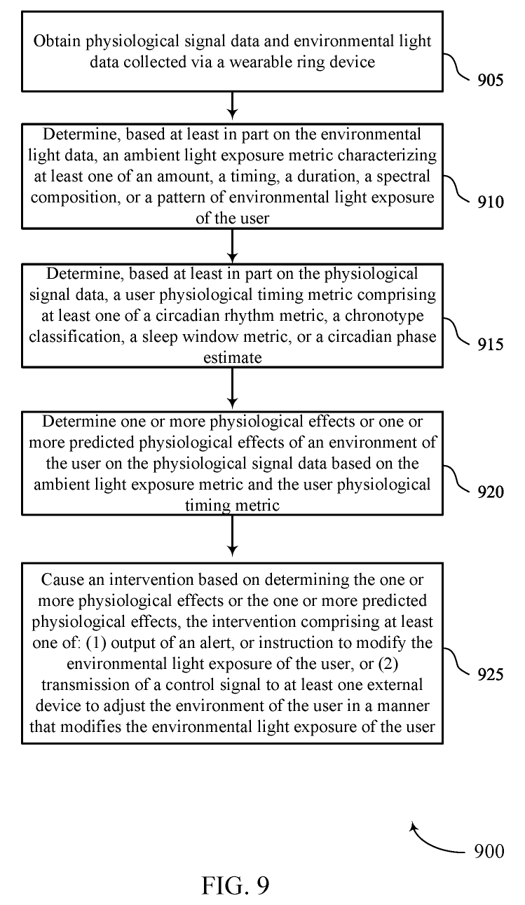

FIG. 9 shows a flowchart illustrating a method 900 that supports techniques for evaluating ambient light exposure and chronotype via wearable devices in accordance with aspects of the present disclosure. The operations of the method 900 may be implemented by a wearable device or its components as described herein. For example, the operations of the method 900 may be performed by a wearable device as described with reference to FIGS. 3 through 8. In some examples, a wearable device may execute a set of instructions to control the functional elements of the wearable device to perform the described functions. Additionally, or alternatively, the wearable device may perform aspects of the described functions using special-purpose hardware.

At 905, the method may include obtaining physiological signal data and environmental light data collected via a wearable ring device. The operations of 905 may be performed in accordance with examples as disclosed herein. In some examples, aspects of the operations of 905 may be performed by a physiological data component 525 as described with reference to FIG. 5.

At 910, the method may include determining, based at least in part on the environmental light data, an ambient light exposure metric characterizing at least one of an amount, a timing, a duration, a spectral composition, or a pattern of environmental light exposure of the user. The operations of 910 may be performed in accordance with examples as disclosed herein. In some examples, aspects of the operations of 910 may be performed by an ambient light component 530 as described with reference to FIG. 5.

At 915, the method may include determining, based at least in part on the physiological signal data, a user physiological timing metric comprising at least one of a circadian rhythm metric, a chronotype classification, a sleep window metric, or a circadian phase estimate. The operations of 915 may be performed in accordance with examples as disclosed herein. In some examples, aspects of the operations of 915 may be performed by a physiological effect component 535 as described with reference to FIG. 5.

At 920, the method may include determine one or more physiological effects or one or more predicted physiological effects of an environment of the user on the physiological signal data based at least in part on the ambient light exposure metric and the user physiological timing metric.

At 925, the method may include cause an intervention based on determining the one or more physiological effects or the one or more predicted physiological effects, the intervention comprising at least one of: (1) output of an alert, or instruction to modify the environmental light exposure of the user, or (2) transmission of a control signal to at least one external device to adjust the environment of the user in a manner that modifies the environmental light exposure of the user. The alert or instruction may include a physical output (e.g., a vibration, audio signal, visual signal, etc.). The approach has the effect of being more accurate, efficient and reliable for informing the user of physiological effects to allow them to take one or more actions to improve their health outcomes. Information may be conveyed to the user via various interactions, including alerts, physical, visual or audio stimuli, feedback, instructions, or information. In other words, the wearable device, user device, or both, may calculate the physiological effects of an environment of the user based on the physiological data, thus making a technical contribution regardless of what use is made of the results. The operations of 925 may be performed in accordance with examples as disclosed herein. In some examples, aspects of the operations of 925 may be performed by a signal component 540 as described with reference to FIG. 5.

It should be noted that the methods described above describe possible implementations, and that the operations and the steps may be rearranged or otherwise modified and that other implementations are possible. Furthermore, aspects from two or more of the methods may be combined.

A method by an apparatus is described. The method may include performing, using one or more light-receiving components of a wearable device, one or more physiological measurements associated with physiological data of a user, performing, using the one or more light-receiving components of the wearable device, one or more measurements of an environmental light exposure of the user while the wearable device is worn by the user, the environmental light exposure representing ambient light incident on the wearable device that is not attributable to light emitted by the wearable device, determining one or more physiological effects of an environment of the user on the physiological data based at least in part on performing the one or more measurements of the environmental light exposure, and based at least in part on determining the one or more physiological effects, causing an intervention comprising at least one of: generating an alert or instruction to modify the environmental light exposure; or transmitting a control signal to at least one external device to adjust the environment of the user in a manner that modifies the environmental light exposure of the user. The at least one external device may be a user device associated with the wearable device and the alert or instruction may display information associated with the one or more physiological effects. Additionally, or alternatively, the at least one external device may be another external device and the control signal may cause an action external to the wearable device to adjust one or more characteristics associated with the environment of the user based at least in part on determining the one or more physiological effects, the one or more actions configured to adjust the environmental light exposure of the user within the environment.

An apparatus is described. The apparatus may include one or more memories storing processor executable code, and one or more processors coupled with the one or more memories. The one or more processors may individually or collectively be operable to execute the code to cause the apparatus to perform, using one or more light-receiving components of a wearable device, one or more physiological measurements associated with physiological data of a user, perform, using the one or more light-receiving components of the wearable device, one or more measurements of an environmental light exposure of the user while the wearable device is worn by the user, the environmental light exposure representing ambient light incident on the wearable device that is not attributable to light emitted by the wearable device, determine one or more physiological effects of an environment of the user on the physiological data based at least in part on performing the one or more measurements of the environmental light exposure, and based at least in part on determining the one or more physiological effects, causing an intervention comprising at least one of: generating an alert or instruction to modify the environmental light exposure; or transmitting a control signal to at least one external device to adjust the environment of the user in a manner that modifies the environmental light exposure of the user.

Another apparatus is described. The apparatus may include means for performing, using one or more light-receiving components of a wearable device, one or more physiological measurements associated with physiological data of a user, means for performing, using the one or more light-receiving components of the wearable device, one or more measurements of an environmental light exposure of the user while the wearable device is worn by the user, the environmental light exposure representing ambient light incident on the wearable device that is not attributable to light emitted by the wearable device, means for determining one or more physiological effects of an environment of the user on the physiological data based at least in part on performing the one or more measurements of the environmental light exposure, and means for based at least in part on determining the one or more physiological effects, causing an intervention comprising at least one of: generating an alert or instruction to modify the environmental light exposure; or transmitting a control signal to at least one external device to adjust the environment of the user in a manner that modifies the environmental light exposure of the user.

A non-transitory computer-readable medium storing code is described. The code may include instructions executable by one or more processors to perform, using one or more light-receiving components of a wearable device, one or more physiological measurements associated with physiological data of a user, perform, using the one or more light-receiving components of the wearable device, one or more measurements of an environmental light exposure of the user while the wearable device is worn by the user, the environmental light exposure representing ambient light incident on the wearable device that is not attributable to light emitted by the wearable device, determine one or more physiological effects of an environment of the user on the physiological data based at least in part on performing the one or more measurements of the environmental light exposure, and based at least in part on determining the one or more physiological effects, causing an intervention comprising at least one of: generating an alert or instruction to modify the environmental light exposure; or transmitting a control signal to at least one external device to adjust the environment of the user in a manner that modifies the environmental light exposure of the user.

Some examples of the method, apparatus, and non-transitory computer-readable medium described herein may further include operations, features, means, or instructions for determining a chronotype of the user based at least in part on the one or more physiological measurements and determining a timing of the environmental light exposure relative to the chronotype based at least in part on the one or more measurements of the environmental light exposure, wherein the one or more physiological effects of the environment may be based at least in part on the timing of the environmental light exposure relative to the chronotype.

Some examples of the method, apparatus, and non-transitory computer-readable medium described herein may further include operations, features, means, or instructions for transmitting one or more signals to a user device associated with the wearable device to cause the user device to display instructions for modifying the timing of the environmental light exposure based at least in part on the chronotype of the user.

Some examples of the method, apparatus, and non-transitory computer-readable medium described herein may further include operations, features, means, or instructions for calculating an ambient light exposure metric of the user based at least in part on the one or more measurements of the environmental light exposure and transmitting one or more signals to a user device associated with the wearable device to cause the user device to display information associated with the ambient light exposure metric.

Some examples of the method, apparatus, and non-transitory computer-readable medium described herein may further include operations, features, means, or instructions for calculating a skin health metric of the user based at least in part on the one or more measurements of the environmental light exposure and transmitting one or more signals to a user device associated with the wearable device to cause the user device to display information associated with the skin health metric.

Some examples of the method, apparatus, and non-transitory computer-readable medium described herein may further include operations, features, means, or instructions for receiving geographical location data associated with a location of the user during a time interval of the one or more measurements of an environmental light exposure and determining an ultraviolet light exposure of the user based at least in part on the geographical location data and the one or more measurements of an environmental light exposure, wherein the skin health metric may be based at least in part on the ultraviolet light exposure.

Some examples of the method, apparatus, and non-transitory computer-readable medium described herein may further include operations, features, means, or instructions for determining at least a first wavelength exposure and a second wavelength exposure of the user based at least in part on the one or more measurements of the environmental light exposure, wherein the one or more physiological effects may be based at least in part on the first wavelength exposure, the second wavelength exposure, or both.

Some examples of the method, apparatus, and non-transitory computer-readable medium described herein may further include operations, features, means, or instructions for determining a location of the user for at least a portion of a time interval associated with the one or more measurements of the environmental light exposure based at least in part on the first wavelength exposure, the second wavelength exposure, or both, wherein the location comprises an indoor location or an outdoor location.

In some examples of the method, apparatus, and non-transitory computer-readable medium described herein, wherein the one or more physiological measurements may be performed using the one or more light-receiving components and one or more light-transmitting components of the wearable device during a first set of measurement occasions at the wearable device and wherein the one or more measurements of the environmental light exposure may be performed during a second set of measurement occasions that may be non-overlapping in time with respect to the first set of measurement occasions.

Some examples of the method, apparatus, and non-transitory computer-readable medium described herein may further include operations, features, means, or instructions for receiving, via a user device associated with the wearable device, a user input indicating one or more relationships for using the at least one external device to modify the environmental light exposure based on the one or more physiological effects, wherein transmitting the control signal may be based at least in part on the user input indicating the one or more relationships.

Some examples of the method, apparatus, and non-transitory computer-readable medium described herein may further include operations, features, means, or instructions for calculating a dosage of vitamin D for the user based at least in part on the one or more measurements of an environmental light exposure and instructing, via a user device associated with the wearable device, the user to intake the dosage of vitamin D.

In some examples of the method, apparatus, and non-transitory computer-readable medium described herein, the at least one external device comprises an external light source, curtains, shades, blinds, or any combination thereof.

In some examples of the method, apparatus, and non-transitory computer-readable medium described herein, the wearable device comprises a wearable ring device, the one or more light-receiving components may be positioned on an inner curved surface of the wearable ring device, and the inner curved surface may be configured to at least partially contact a tissue of the user when the wearable ring device may be worn by the user.

In some examples of the method, apparatus, and non-transitory computer-readable medium described herein, the wearable device comprises a wearable ring device, the one or more light-receiving components may be positioned on an inner curved surface of the wearable ring device, and the inner curved surface may be configured to at least partially contact a tissue of the user when the wearable ring device may be worn by the user.

Aspects of the present disclosure are described below. Each item below corresponds to an aspect of the disclosure, and each possible combination of any of the items corresponds to an aspect of the disclosure. When items are combined, two or more instances of an element with the same name are to be understood as referring to the same element, even if the element is introduced by the word “a” more than once. Each possible combination of any features from the items below with any features described above also forms part of the disclosure:

Aspect 1: A method, comprising: performing, using one or more light-receiving components of a wearable device, one or more physiological measurements associated with physiological data of a user; performing, using the one or more light-receiving components of the wearable device, one or more measurements of an environmental light exposure of the user while the wearable device is worn by the user, the environmental light exposure representing ambient light incident on the wearable device that is not attributable to light emitted by the wearable device; determining one or more physiological effects of an environment of the user on the physiological data based at least in part on performing the one or more measurements of the environmental light exposure; and based at least in part on determining the one or more physiological effects, causing an intervention comprising at least one of: generating an alert or instruction to modify the environmental light exposure; or transmitting a control signal to at least one external device to adjust the environment of the user in a manner that modifies the environmental light exposure of the user.

Aspect 2: The method of aspect 1, further comprising: determining a chronotype of the user based at least in part on the one or more physiological measurements; and determining a timing of the environmental light exposure relative to the chronotype based at least in part on the one or more measurements of the environmental light exposure, wherein the one or more physiological effects of the environment are based at least in part on the timing of the environmental light exposure relative to the chronotype.

Aspect 3: The method of aspect 2, further comprising: transmitting one or more signals to a user device associated with the wearable device to cause the user device to display instructions for modifying the timing of the environmental light exposure based at least in part on the chronotype of the user.

Aspect 4: The method of any of aspects 1 to 3, further comprising: calculating an ambient light exposure metric of the user based at least in part on the one or more measurements of the environmental light exposure; and transmitting one or more signals to a user device associated with the wearable device to cause the user device to display information associated with the ambient light exposure metric.

Aspect 5: The method of any of aspects 1 to 4, further comprising: calculating a skin health metric of the user based at least in part on the one or more measurements of the environmental light exposure; and transmitting one or more signals to a user device associated with the wearable device to cause the user device to display information associated with the skin health metric.

Aspect 6: The method of aspect 5, further comprising: receiving geographical location data associated with a location of the user during a time interval of the one or more measurements of the environmental light exposure; and determining an ultraviolet light exposure of the user based at least in part on the geographical location data and the one or more measurements of the environmental light exposure, wherein the skin health metric is based at least in part on the ultraviolet light exposure.

Aspect 7: The method of any of aspects 1 to 6, further comprising: determining at least a first wavelength exposure and a second wavelength exposure of the user based at least in part on the one or more measurements of the environmental light exposure, wherein the one or more physiological effects are based at least in part on the first wavelength exposure, the second wavelength exposure, or both.

Aspect 8: The method of aspect 7, further comprising: determining a location of the user for at least a portion of a time interval associated with the one or more measurements of the environmental light exposure based at least in part on the first wavelength exposure, the second wavelength exposure, or both, wherein the location comprises an indoor location or an outdoor location.

Aspect 9: The method of any of aspects 1 to 8, wherein the one or more physiological measurements are performed using the one or more light-receiving components and one or more light-transmitting components of the wearable device during a first set of measurement occasions at the wearable device, and wherein the one or more measurements of the environmental light exposure are performed during a second set of measurement occasions that are non-overlapping in time with respect to the first set of measurement occasions.

Aspect 10: The method of any of aspects 1 to 9, further comprising: receiving, via a user device associated with the wearable device, a user input indicating one or more relationships for using the at least one external device to modify the environmental light exposure based on the one or more physiological effects, wherein transmitting the control signal is based at least in part on the user input indicating the one or more relationships.

Aspect 11: The method of any of aspects 1 to 10, further comprising: calculating a dosage of vitamin D for the user based at least in part on the one or more measurements of the environmental light exposure; and instructing, via a user device associated with the wearable device, the user to intake the dosage of vitamin D.

Aspect 12: The method of any of aspects 1 to 11, wherein the at least one external device comprises an external light source, curtains, shades, blinds, or any combination thereof.

Aspect 13: The method of any of aspects 1 to 12, wherein the wearable device comprises a wearable ring device, wherein the one or more light-receiving components are positioned on an inner curved surface of the wearable ring device, wherein the inner curved surface is configured to at least partially contact a tissue of the user when the wearable ring device is worn by the user.

Aspect 14: An apparatus, comprising: one or more memories storing processor executable code; and one or more processors coupled with the one or more memories and individually or collectively operable to execute the code to cause the apparatus to: perform, using one or more light-receiving components of a wearable device, one or more physiological measurements associated with physiological data of a user; perform, using the one or more light-receiving components of the wearable device, one or more measurements of an environmental light exposure of the user while the wearable device is worn by the user, the environmental light exposure representing ambient light incident on the wearable device that is not attributable to light emitted by the wearable device; determine one or more physiological effects of an environment of the user on the physiological data based at least in part on performing the one or more measurements of the environmental light exposure; and based at least in part on determining the one or more physiological effects, causing an intervention comprising at least one of: generating an alert or instruction to modify the environmental light exposure; or transmitting a control signal to adjust the environment of the user in a manner that modifies the environmental light exposure of the user.

Aspect 15: The apparatus of aspect 14, wherein the one or more processors are further operable to execute the code to cause the apparatus to: determine a chronotype of the user based at least in part on the one or more physiological measurements; and determine a timing of the environmental light exposure relative to the chronotype based at least in part on the one or more measurements of the environmental light exposure, wherein the one or more physiological effects of the environment are based at least in part on the timing of the environmental light exposure relative to the chronotype.

Aspect 16: The apparatus of aspects 14 to 15, wherein the one or more processors are further operable to execute the code to cause the apparatus to: transmit one or more signals to a user device associated with the wearable device to cause a user device to display instructions for modifying the timing of the environmental light exposure based at least in part on the chronotype of the user.

Aspect 17: The apparatus of aspects 14 to 16, wherein the one or more processors are further operable to execute the code to cause the apparatus to: calculate an ambient light exposure metric of the user based at least in part on the one or more measurements of the environmental light exposure; and transmit one or more signals to a user device associated with the wearable device to cause the user device to display information associated with the ambient light exposure metric.

Aspect 18: The apparatus of aspects 14 to 17, wherein the one or more processors are further operable to execute the code to cause the apparatus to: calculate a skin health metric of the user based at least in part on the one or more measurements of the environmental light exposure; and transmit one or more signals to a user device associated with the wearable device to cause the user device to display information associated with the skin health metric.

Aspect 19: The apparatus of aspects 14 to 18, wherein the one or more processors are further operable to execute the code to cause the apparatus to: receive geographical location data associated with a location of the user during a time interval of the one or more measurements of the environmental light exposure; and determine an ultraviolet light exposure of the user based at least in part on the geographical location data and the one or more measurements of the environmental light exposure, wherein the skin health metric is based at least in part on the ultraviolet light exposure.

Aspect 20: A wearable system, comprising: a wearable device configured to be worn by a user, the wearable device comprising: one or more physiological sensors configured to generate physiological signal data of the user; one or more light-sensing components configured to generate environmental light data indicative of ambient light incident on the wearable device while worn by the user; and a communication interface; and one or more processors operatively coupled to at least one of the wearable device or a user device communicatively coupled to the wearable device, the one or more processors configured to: obtain the physiological signal data and the environmental light data; determine, based at least in part on the environmental light data, an ambient light exposure metric characterizing at least one of an amount, timing, duration, spectral composition, or pattern of environmental light exposure of the user; determine, based at least in part on the physiological signal data, a user physiological timing metric comprising at least one of a circadian rhythm metric, a chronotype classification, a sleep window metric, or a circadian phase estimate; determine one or more physiological effects or one or more predicted physiological effects of the environment of the user on the physiological data based at least in part on the ambient light exposure metric and the user physiological timing metric; and based at least in part on determining the one or more physiological effects or the one or more predicted physiological effects, cause an intervention comprising at least one of: output of an alert or instruction to modify the environmental exposure of the user; or transmission of a control signal to at least one external device to adjust the environment of the user in a manner that modifies the environmental light exposure of the user.

Aspect 21: A computer readable medium comprising computer-executable instructions configured to, when executed by a processor, perform the method of any of aspects 1 to 20.

Aspect 21: A wearable device comprising one or more light receiving components, a processor and a memory storing the computer readable medium of aspect 20.

Aspect 22: The wearable device of aspect 21, wherein the wearable device comprises a wearable ring device, wherein the one or more light-receiving components are positioned on an inner curved surface of the wearable ring device, wherein the inner curved surface is configured to at least partially contact a tissue of the user when the wearable ring device is worn by the user.

Aspect 23: A wearable device comprising: one or more light-receiving components configured to perform one or more physiological measurements associated with physiological data of a user and one or more measurements of an environmental light exposure of the user; means for determining one or more physiological effects of an environment of the user on the physiological data based at least in part on performing the one or more measurements of the environmental light exposure; and means for transmitting one or more signals to an external device to cause the external device to perform an action based on the physiological effect.

Aspect 24: The wearable device of aspect 23 configured to perform the method of any of claims 1 to 20.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A method, comprising:
performing, using one or more light-receiving components of a wearable device, one or more physiological measurements associated with physiological data of a user;
performing, using the one or more light-receiving components of the wearable device, one or more measurements of an environmental light exposure of the user while the wearable device is worn by the user, the environmental light exposure representing ambient light incident on the wearable device that is not attributable to light emitted by the wearable device;
determining one or more physiological effects of an environment of the user on the physiological data based at least in part on performing the one or more measurements of the environmental light exposure; and
based at least in part on determining the one or more physiological effects, causing an intervention comprising at least one of:
generating an alert or instruction to modify the environmental light exposure; or
transmitting a control signal to at least one external device to adjust the environment of the user in a manner that modifies the environmental light exposure of the user.

2. The method of claim 1, further comprising:
determining a chronotype of the user based at least in part on the one or more physiological measurements; and
determining a timing of the environmental light exposure relative to the chronotype based at least in part on the one or more measurements of the environmental light exposure, wherein the one or more physiological effects of the environment are based at least in part on the timing of the environmental light exposure relative to the chronotype.

3. The method of claim 2, wherein generating the alert or instruction further comprises:
transmitting one or more signals to a user device associated with the wearable device to cause the user device to display instructions for modifying the timing of the environmental light exposure based at least in part on the chronotype of the user.

4. The method of claim 1, further comprising:
calculating an ambient light exposure metric of the user based at least in part on the one or more measurements of the environmental light exposure; and
transmitting one or more signals to a user device associated with the wearable device to cause the user device to display information associated with the ambient light exposure metric.

5. The method of claim 1, further comprising:
calculating a skin health metric of the user based at least in part on the one or more measurements of the environmental light exposure; and
transmitting one or more signals to a user device associated with the wearable device to cause the user device to display information associated with the skin health metric.

6. The method of claim 5, further comprising:
receiving geographical location data associated with a location of the user during a time interval of the one or more measurements of the environmental light exposure; and
determining an ultraviolet light exposure of the user based at least in part on the geographical location data and the one or more measurements of the environmental light exposure, wherein the skin health metric is based at least in part on the ultraviolet light exposure.

7. The method of claim 1, further comprising:
determining at least a first wavelength exposure and a second wavelength exposure of the user based at least in part on the one or more measurements of the environmental light exposure, wherein the one or more physiological effects are based at least in part on the first wavelength exposure, the second wavelength exposure, or both.

8. The method of claim 7, further comprising:
determining a location of the user for at least a portion of a time interval associated with the one or more measurements of the environmental light exposure based at least in part on the first wavelength exposure, the second wavelength exposure, or both, wherein the location comprises an indoor location or an outdoor location.

9. The method of claim 1, wherein the one or more physiological measurements are performed using the one or more light-receiving components and one or more light-transmitting components of the wearable device during a first set of measurement occasions at the wearable device, and wherein the one or more measurements of the environmental light exposure are performed during a second set of measurement occasions that are non-overlapping in time with respect to the first set of measurement occasions.

10. The method of claim 1, further comprising:
receiving, via a user device associated with the wearable device, a user input indicating one or more relationships for using the at least one external device to modify the environmental light exposure based on the one or more physiological effects, wherein transmitting the control signal is based at least in part on the user input indicating the one or more relationships.

11. The method of claim 1, further comprising:
calculating a dosage of vitamin D for the user based at least in part on the one or more measurements of the environmental light exposure; and
instructing, via a user device associated with the wearable device, the user to intake the dosage of vitamin D.

12. The method of claim 1, wherein the at least one external device comprises an external light source, curtains, shades, blinds, or any combination thereof.

13. The method of claim 1, wherein the wearable device comprises a wearable ring device, wherein the one or more light-receiving components are positioned on an inner curved surface of the wearable ring device, wherein the inner curved surface is configured to at least partially contact a tissue of the user when the wearable ring device is worn by the user.

14. An apparatus, comprising:
one or more memories storing processor executable code; and
one or more processors coupled with the one or more memories and individually or collectively operable to execute the code to cause the apparatus to:
perform, using one or more light-receiving components of a wearable device, one or more physiological measurements associated with physiological data of a user;
perform, using the one or more light-receiving components of the wearable device, one or more measurements of an environmental light exposure of the user while the wearable device is worn by the user, the environmental light exposure representing ambient light incident on the wearable device that is not attributable to light emitted by the wearable device;
determine one or more physiological effects of an environment of the user on the physiological data based at least in part on performing the one or more measurements of the environmental light exposure; and
based at least part on determining the one or more physiological effects, cause an intervention comprising at least one of:
generate an alert or instruction to modify the environmental light exposure; or
transmit a control signal to at least one external device to adjust the environment of the user in a manner that modifies the environmental light exposure of the user.

15. The apparatus of claim 14, wherein the one or more processors are further operable to execute the code to cause the apparatus to:
determine a chronotype of the user based at least in part on the one or more physiological measurements; and
determine a timing of the environmental light exposure relative to the chronotype based at least in part on the one or more measurements of the environmental light exposure, wherein the one or more physiological effects of the environment are based at least in part on the timing of the environmental light exposure relative to the chronotype.

16. The apparatus of claim 15, wherein, to generate the alert or instruction, the one or more processors are further operable to execute the code to cause the apparatus to:
transmit one or more signals to a user device associated with the wearable device to cause a user device to display instructions for modifying the timing of the environmental light exposure based at least in part on the chronotype of the user.

17. The apparatus of claim 14, wherein the one or more processors are further operable to execute the code to cause the apparatus to:
calculate an ambient light exposure metric of the user based at least in part on the one or more measurements of the environmental light exposure; and
transmit one or more signals to a user device associated with the wearable device to cause the user device to display information associated with the ambient light exposure metric.

18. The apparatus of claim 14, wherein the one or more processors are further operable to execute the code to cause the apparatus to:
calculate a skin health metric of the user based at least in part on the one or more measurements of the environmental light exposure; and
transmit one or more additional signals to a user device associated with the wearable device to cause the user device to display information associated with the skin health metric.

19. The apparatus of claim 18, wherein the one or more processors are further operable to execute the code to cause the apparatus to:
receive geographical location data associated with a location of the user during a time interval of the one or more measurements of the environmental light exposure; and
determine an ultraviolet light exposure of the user based at least in part on the geographical location data and the one or more measurements of the environmental light exposure, wherein the skin health metric is based at least in part on the ultraviolet light exposure.

20. A wearable system, comprising:
a wearable device configured to be worn by a user, the wearable device comprising:
one or more physiological sensors configured to generate physiological signal data of the user;
one or more light-sensing components configured to generate environmental light data indicative of ambient light incident on the wearable device while worn by the user; and
a communication interface; and
one or more processors operatively coupled to at least one of the wearable device or a user device that is communicatively coupled to the wearable device, the one or more processors configured to:
obtain the physiological signal data and the environmental light data;
determine, based at least in part on the environmental light data, an ambient light exposure metric characterizing at least one of an amount, a timing, a duration, a spectral composition, or a pattern of environmental light exposure of the user;
determine, based at least in part on the physiological signal data, a user physiological timing metric comprising at least one of a circadian rhythm metric, a chronotype classification, a sleep window metric, or a circadian phase estimate;
determine one or more physiological effects or one or more predicted physiological effects of an environment of the user on the physiological signal data based at least in part on the ambient light exposure metric and the user physiological timing metric; and
based at least in part on determining the one or more physiological effects or the one or more predicted physiological effects, cause an intervention comprising at least one of:
output of an alert, or instruction to modify the environmental light exposure of the user; or transmission of a control signal to at least one external device to adjust the environment of the user in a manner that modifies the environmental light exposure of the user.
